(19) United States
(12) Patent Application Publication
(10) Pub. No.: US 2022/0251187 A1
(43) Pub. Date: Aug. 11, 2022

(54) P2X7 RECEPTOR TARGETED THERAPY

(71) Applicant: BIOSCEPTRE (AUST) PTY LTD, New South Wales (AU)

(72) Inventors: Shaun MCNULTY, Cambridge (GB); Romain LARA, Cambridge (GB); Chris OLIPHANT, Cambridge (GB); Simon GILBERT, Cambridge (GB); Ermira LLESHI, Cambridge (GB)

(21) Appl. No.: 17/618,849

(22) PCT Filed: Jul. 24, 2020
(86) PCT No.: PCT/GB2020/051791

§ 371 Date: Dec. 13, 2021

(30) Foreign Application Priority Data
Jul. 26, 2019 (AU) 2019902672

(51) Int. Cl. C07K 16/28; A61K 31/7105; A61K 35/17; A61K 38/17; A61K 31/513; A61K 31/7068; A61K 31/704; A61K 31/475; A61K 31/4745; A61K 31/407; A61K 31/337; A61K 31/517; A61K 33/243; A61K 31/675; A61P 35/00

(52) U.S. Cl. CPC C07K 16/28; C07K 2319/30; A61K 35/17; A61K 38/1774; A61K 31/513; A61K 31/7068; A61K 31/704; A61K 31/475; A61K 31/4745; A61K 31/407; A61K 31/337; A61K 31/517; A61K 33/243; A61K 31/675; A61P 35/00; C07K 2317/76; C07K 2317/622; A61K 31/7105

(57) ABSTRACT

The invention relates to methods of treating cancer, particular cancers which have developed a resistance to chemotherapeutics. Particularly, the invention relates to a method of treating cancer in an individual who has not responded, or no longer responds, to chemotherapy, the method comprising providing an individual who has not responded, or no longer responds, to a chemotherapeutic agent; providing in the individual a whole antibody or a fragment thereof including a variable domain for binding to a P2X7 receptor that is expressed by the individual; wherein the P2X7 receptor has an impaired response to ATP such that it is unable to form an apoptotic pore under normal physiological conditions, thereby treating cancer in the individual.

### FIELD OF THE INVENTION

The invention relates to methods of treating cancer, particular cancers which have developed a resistance to chemotherapeutics.

### CROSS-REFERENCE TO EARLIER APPLICATION

This application claims priority from Australian provisional application no. 2019902672 the entire contents of which is incorporated by reference in its entirety.

### BACKGROUND OF THE INVENTION

Despite improvements in therapies for the treatment of cancer, the cancer mortality rate worldwide remains high and strategies to prevent cancer recurrence are still needed. Current therapeutic strategies against cancer frequently result in treatment failure, often due to the development of multiple malignancies and/or resistance to chemotherapy and radiotherapy.

The development of chemoresistance is a persistent problem during chemotherapy treatment. For instance, the conventional treatment of acute myeloid leukemia (AML) comprises the combined administration of cytarabine with an anthracycline, such as daunorubicin. 5-year overall survival rate is 40% in young adults and around 10% for elderly patients. Response rates dramatically vary with ageing, from 40% to 55% in patients older than 60 years and from 24% to 33% in patients older than 70 years. These data underline the need for new approaches both to reduce dosage regimens of antineoplastic agents to treat chemosensitive tumours and by-pass resistance of chemoresistant tumours to antineoplastic agents.

Various hypotheses have been proposed to account for the phenomenon of chemoresistance. The hypotheses include altered transport of the drug across the plasma membrane, genetic responses, enhanced DNA repair, alteration in target molecules, access to target cells, metabolic effects and growth factors. Recently, small pumps on the surface of cancer cells that actively move chemotherapy drugs from inside the cell to the outside have been identified. Research on p-glycoprotein and other such chemotherapy efflux pumps is currently ongoing. Medications inhibiting the function of p-glycoprotein have been explored to enhance the efficacy of chemotherapy. However, this approach failed during clinical evaluation. It is nevertheless increasingly recognized that the causes of chemoresistance and relapse reside within a small number of cells that undergo further mutations as part of the transformation processes acting in cancer.

The recent renaissance in understanding the role of clonal evolution in tumourigenesis illuminates the challenge of acquired resistance and has led to two major models of tumourigenesis—the cancer stem cell and clonal evolution models. These are not necessarily mutually exclusive and can be complementary. Both indicate that the main obstacle to durable cures is cancer heterogeneity. However, existing anti-cancer therapy largely fails to account for either model. Recent advances in targeted therapy are promising, but since cancer heterogeneity and evolution present multiple, moving targets, development of resistance is common. Unfortunately, little progress has been made in targeting the chemoresistant cells responsible for relapse.

There is a need for new and/or improved cancer therapy regimens and in particular therapies to overcome chemoresistance of cancer cells or to increase the sensitivity of cancer cells to non-target therapies such as chemotherapy and/or radiotherapy.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method of treating cancer in an individual who has not responded, or no longer responds, to chemotherapy and/or radiotherapy, the method comprising
providing an individual who has not responded, or no longer responds, to a chemotherapeutic agent and/or radiotherapy;
administering a P2X7 receptor targeted therapy to the individual;

wherein the P2X7 receptor has an impaired response to ATP such that it is unable to form an apoptotic pore under normal physiological conditions,

thereby treating cancer in the individual.

In any aspect of the present invention, the individual no longer responds to one or more chemotherapeutic agents selected from the group consisting of oxazaphosphorines, topoisomerase I inhibitors, topoisomerase II inhibitors, thymidylate synthase inhibitors, proteasome inhibitors, antifolates, nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, purine antagonists, antimetabolites, antibiotics, epipodophyllotoxins, platinum based agents, ribonucleotide reductase inhibitors, vinca alkaloids, substituted ureas, hydrazine derivatives, adrenocortical suppressants, endostatin, camptothecins, oxaliplatin, doxorubicin and doxorubicin analogs, antibiotics, L-asparaginase, tyrosine kinase inhibitors, or derivatives or variants thereof.

Preferably, the one or more chemotherapeutic agents are selected from the group consisting of doxorubicin, cisplatin, vincristine, dacarbazine (DTIC), cyclophosphamide, CPT-11, oxaliplatin, gemcitabine and 5-fluorouracil/leucovorin.

Alternatively, the one or more chemotherapeutic agents are selected from the group consisting of 5FU, Folinic acid, Bleomycin, Etoposide, Cisplatin, Capecitabin, Oxaliplatin, Dacarbazine, Cyclophosphamide, Vincristine, Doxorubicin, Irinotecan, Gemcitabine, Mitomycin-C, Gemcitabine, Carboplatin, Paclitaxel, pemetrexed, hydroxyethyl-chloroethyl nitrosourea (HeCNU), Tamoxifen, Methotrexat, Epirubicin, Vindesine, Erlotinib, Bevacizumab, Cetuximab.

In any aspect of the present invention, the individual has any cancer as described herein. Preferably, the cancer is selected from the group consisting of colorectal cancer, testicular cancer, sarcoma, melanoma, bladder cancer, pancreatic cancer, small cell lung cancer, non-small cell lung cancer, ovarian cancer, and breast cancer.

In any aspect of the present invention, the individual has colon cancer.

In any aspect of the present invention, the individual has ovarian cancer.

In any aspect, the P2X7 receptor targeted therapy results in a reduction in the viability of a cancer cell expressing a P2X7 receptor that has an impaired response to ATP such that it is unable to form an apoptotic pore under normal physiological conditions. The P2X7 receptor targeted therapy may be a direct or indirect inhibitor of P2X7 receptor activity or expression level, wherein the P2X7 receptor has an impaired response to ATP such that it is unable to form an apoptotic pore under normal physiological conditions. For example, the inhibitor may be an interfering RNA capable of reducing the level of P2X7 receptor in a cancer cell.

An inhibitor of a P2X7 receptor may be selected from the group consisting of a small molecule, an antibody, a peptide or an interfering RNA.

In any aspect, the inhibitor of a P2X7 receptor may be a molecule that induces an immune response in the individual to a P2X7 receptor that is expressed by the individual, preferably a P2X7 receptor expressed on a cancer cell.

In another aspect, the present invention provides a method of treating cancer in an individual who has not responded, or no longer responds, to chemotherapy and/or radiotherapy, the method comprising
providing an individual who has not responded, or no longer responds, to a chemotherapeutic agent and/or radiotherapy;
providing in the individual a whole antibody or a fragment thereof comprising a variable domain for binding to a P2X7 receptor that is expressed by the individual;

wherein the P2X7 receptor has an impaired response to ATP such that it is unable to form an apoptotic pore under normal physiological conditions,

thereby treating cancer in the individual.

In any aspect, administering a P2X7 receptor targeted therapy to the individual comprises providing in the individual a whole antibody or a fragment thereof comprising a variable domain for binding to a P2X7 receptor that is expressed by the individual, wherein the P2X7 receptor has an impaired response to ATP such that it is unable to form an apoptotic pore under normal physiological conditions.

In any aspect of the present invention, the antibody fragment is selected from the group consisting of a dAb, Fab, Fd, Fv, F(ab′)2, scFv or any other antibody fragment format described herein.

In any aspect of the present invention, the antibody or fragment thereof does not bind to functional P2X7 receptors (i.e. P2X7 receptors that do not have an impaired response to ATP and are therefore capable of forming an apoptotic pore under normal physiological conditions). Preferably, the antibody or fragment thereof binds to the amino acid sequence as set forth in any one of SEQ ID NOs: 1 to 11, more preferably, SEQ ID NO: 2 to 5.

In any aspect of the present invention, the antibody or fragment thereof comprises the amino acid sequences of any antibody described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. Nos. 8,293,491, 8,658,385), PCT/AU2009/000869 (or in any one of the corresponding U.S. Pat. Nos. 8,597,643, 9,328,155 or 10,238,716) and PCT/AU2010/001070 (or in any one of the corresponding U.S. Pat. Nos. 9,127,059, 9,688,771, or 10,053,508), the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In another aspect, the present invention provides a method of treating cancer in an individual who has not responded, or no longer responds, to chemotherapy and/or radiotherapy, the method comprising
providing an individual who has not responded, or no longer responds, to a chemotherapeutic agent and/or radiotherapy;
providing in the individual a cell-based therapy that targets P2X7 receptor expressing cancer cells;
wherein the P2X7 receptor has an impaired response to ATP such that it is unable to form an apoptotic pore under normal physiological conditions,

thereby treating cancer in the individual.

In any aspect, a cell based therapy that targets P2X7 receptor expressing cancer cells may be a cytotoxic cell that has the capacity to bind to a P2X7 receptor expressing cancer cell, for example a CAR-T cell.

In this aspect, the cytotoxic cell, preferably a CAR-T cell, expresses a chimeric antigen receptor including an antigen-recognition domain and a signalling domain, wherein the antigen-recognition domain recognises a dysfunctional or non-functional P2X7 receptor (i.e. a P2X7 receptor has an impaired response to ATP such that it is unable to form an apoptotic pore under normal physiological conditions). Typically, the dysfunctional or non-functional P2X7 receptor has a reduced capacity to bind ATP compared to an ATP-binding capacity of a wild-type (functional) P2X7 receptor. The dysfunctional or non-functional P2X7 receptor may have a conformational change that renders the receptor dysfunctional or non-functional.

In this aspect, the antigen-recognition domain may recognise an epitope that includes proline at amino acid position 210 of the P2X7 receptor.

In this aspect, the antigen-recognition domain may recognise an epitope that includes one or more amino acid residues spanning from glycine at amino acid position 200 to cysteine at amino acid position 216 of the dysfunctional P2X7 receptor.

In this aspect, the antigen-recognition domain may comprise amino acid sequence homology to the amino acid sequence of an antibody, or a fragment thereof, that binds to the dysfunctional or non-functional P2X7 receptor, including any antibody, or fragment thereof, as described herein.

In this aspect, the antigen-recognition domain may include amino acid sequence homology to the amino acid sequence of a fragment-antigen binding (Fab) portion, a single-chain variable fragment (scFv), or a single-antibody domain (dAb) of an antibody that binds to a dysfunctional or non-functional P2X7 receptor.

In this aspect, the antigen-recognition domain may include amino acid sequence homology to the amino acid sequence of a multivalent single-chain variable fragment (scFv) that binds to a dysfunctional or non-functional P2X7 receptor. The multivalent single-chain variable fragment (scFv) may be di-valent or tri-valent scFv.

In this aspect, the signalling domain may include a portion derived from an activation receptor. Typically, the activation receptor is a member of the CD3 co-receptor complex. Preferably, the portion derived from the CD3 co-receptor complex is CD3-zeta. Alternatively, the activation receptor is an Fc receptor and then preferably the portion derived from the Fc receptor is Fc epsilon RI or Fc gamma RI.

In this aspect, the signalling domain may include a portion derived from a co-stimulatory receptor.

In this aspect, the signalling domain may include a portion derived from an activation receptor and a portion derived from a co-stimulatory receptor.

In this aspect, the co-stimulatory receptor may be selected from the group consisting of CD27, CD28, CD30, CD40, DAP10, OX40, 4-1 BB (CD137) and ICOS.

In this aspect, the cytotoxic cell any one of:
a leukocyte,
a Peripheral Blood Mononuclear Cell (PBMC),
a lymphocyte,
a T cell,
a CD4+ T cell,
a CD8+ T cell,
a natural killer cell, or
a natural killer T cell.

In another aspect, the present invention also provides a method of treating cancer in an individual who has not responded, or no longer responds, to chemotherapy and/or radiotherapy, the method comprising
providing an individual who has not responded, or no longer responds, to chemotherapy and/or radiotherapy;
forming an immune response in the individual to a P2X7 receptor that is expressed by the individual;
   wherein the P2X7 receptor has an impaired response to ATP such that it is unable to form an apoptotic pore under normal physiological conditions,

thereby treating cancer in the individual.

In any aspect of the present invention, the immune response is formed by providing an immunogen in the individual in the form of a P2X7 receptor, or a fragment of a P2X7 receptor that is capable of inducing an immune response to a P2X7 receptor in the individual, wherein the P2X7 receptor has an impaired response to ATP such that it is unable to form an apoptotic pore under normal physiological conditions. Preferably, the fragment of a P2X7 receptor has an amino acid sequence selected from the group consisting of SEQ ID NO: 1 to 11. More preferably, SEQ ID NO: 2 to 5.

The immunogen may contain at least one sequence that is capable of being presented on a major histocompatibility complex class II molecule and/or is capable of interacting with a T or B-cell receptor or a B-cell membrane bound-immunoglobulin.

According to the invention, the individual is human, in which case the immunogen is provided in the form of a human P2X7 receptor, or fragment thereof that is capable of inducing an immune response to a P2X7 receptor.

Typically the immune response that is formed in the individual is specific for a P2X7 receptor that has an impaired response to ATP such that it is unable to form an apoptotic pore under normal physiological conditions. In which case, antibodies or cellular components that are reactive with non-functional P2X7 receptors (i.e. with one or more sites unable to bind ATP), but not reactive with functional P2X7 receptors (i.e. ATP binding receptors) are formed in the individual.

In any aspect of the present invention, the immunogen is provided in an initial administration to the individual, thereby forming a response that includes IgM production in the individual.

In any aspect of the present invention, immunogen is provided in an initial administration to the individual, thereby forming a response that includes IgM production, and at a later time, in a further administration to the initial administration, thereby forming a response that includes IgG production.

The immune response may be a humoral and/or cellular response.

A humoral response may include the transformation of B-cells into plasma cells, which secrete antibody, Th2 activation and cytokine production, germinal centre formation and isotype switching, affinity maturation of B-cells and/or memory cell generation.

A cellular response may include activating antigen-specific cytotoxic T-lymphocytes, activating macrophages and natural killer cells and/or stimulating cells to secrete cytokines.

The humoral and/or cellular response formed in the individual may treat or ameliorate a cancer in the individual, or minimise the progression of cancer in the individual.

In another aspect, the present invention also provides a method of treating cancer in an individual, the method comprising
administering a chemotherapeutic agent and/or radiotherapy to the individual in whom the cancer is to be treated; and
administering to the individual a whole antibody or a fragment thereof including a variable domain for binding to a P2X7 receptor that is expressed by the individual;

wherein the P2X7 receptor has an impaired response to ATP such that it is unable to form an apoptotic pore under normal physiological conditions,

thereby treating cancer in the individual.

In another aspect, the present invention also provides a method of treating cancer in an individual, the method comprising
providing an individual who has responded to chemotherapy and/or radiotherapy; and
administering to the individual a whole antibody or a fragment thereof including a variable domain for binding to a P2X7 receptor that is expressed by the individual;

wherein the P2X7 receptor has an impaired response to ATP such that it is unable to form an apoptotic pore under normal physiological conditions,

thereby treating cancer in the individual.

In the present aspect, the chemotherapeutic agent and/or radiotherapy may be administered simultaneously with the antibody or fragment thereof. In one embodiment, the chemotherapy and antibody or fragment thereof are administered simultaneously. In another embodiment, the radiotherapy and antibody or fragment thereof are administered simultaneously.

In the present aspect, the chemotherapeutic agent and/or radiotherapy may be administered sequentially to the antibody or fragment thereof.

In the present aspect, the chemotherapeutic agent may be administered prior to the antibody or fragment thereof.

In the present aspect, the chemotherapeutic agent may be any described herein. Preferably, the chemotherapeutic agent is selected from the group consisting of oxazaphosphorines, topoisomerase I inhibitors, topoisomerase II inhibitors, thymidylate synthase inhibitors, proteasome inhibitors, antifolates, nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, purine antagonists, antimetabolites, antibiotics, epipodophyllotoxins, platinum based agents, ribonucleotide reductase inhibitors, vinca alkaloids, substituted ureas, hydrazine derivatives, adrenocortical suppressants, endostatin, camptothecins, oxaliplatin, doxorubicin and doxorubicin analogs, antibiotics, L-asparaginase, tyrosine kinase inhibitors, or derivatives or variants thereof.

Preferably, the chemotherapeutic agent is selected from the group consisting of doxorubicin, cisplatin, vincristine, dacarbazine (DTIC), cyclophosphamide, CPT-11, oxaliplatin, gemcitabine and 5-fluorouracil/leucovorin.

Alternatively, the chemotherapeutic agent is selected from the group consisting of 5FU, Folinic acid, Bleomycin, Etoposide, Cisplatin, Capecitabin, Oxaliplatin, Dacarbazine, Cyclophosphamide, Vincristine, Doxorubicin, Irinotecan, Gemcitabine, Mitomycin-C, Gemcitabine, Carboplatin, Paclitaxel, pemetrexed, hydroxyethyl-chloroethyl nitrosourea (HeCNU), Tamoxifen, Methotrexat, Epirubicin, Vindesine, Erlotinib, Bevacizumab, Cetuximab.

In another aspect, the present invention also provides a method of treating cancer in an individual, the method comprising
administering a chemotherapeutic agent and/or radiotherapy to the individual in whom the cancer is to be treated; and
forming an immune response in the individual to a P2X7 receptor that is expressed by the individual;

wherein the P2X7 receptor has an impaired response to ATP such that it is unable to form an apoptotic pore under normal physiological conditions,

thereby treating cancer in the individual.

In another aspect, the present invention also provides a method of treating cancer in an individual, the method comprising
providing an individual who has responded to chemotherapy and/or radiotherapy; and
forming an immune response in the individual to a P2X7 receptor that is expressed by the individual;

wherein the P2X7 receptor has an impaired response to ATP such that it is unable to form an apoptotic pore under normal physiological conditions,

thereby treating cancer in the individual.

In any aspect of the present invention, the immune response is formed by providing an immunogen in the individual in the form of a P2X7 receptor, or a fragment of a P2X7 receptor that is capable of inducing an immune response to a P2X7 receptor in the individual, wherein the P2X7 receptor has an impaired response to ATP such that it is unable to form an apoptotic pore under normal physiological conditions. Preferably, the fragment of a P2X7 receptor has an amino acid sequence selected from the group consisting of SEQ ID NO: 1 to 11, more preferably, SEQ ID NO: 2 to 5.

In the present aspect, the chemotherapeutic agent and the immunogen may be administered simultaneously.

In the present aspect, the chemotherapeutic agent and the immunogen may be administered sequentially.

In the present aspect, the chemotherapeutic agent may be administered prior to the immunogen.

In the present aspect, the chemotherapeutic agent may be any described herein. Preferably, the chemotherapeutic agent is selected from the group consisting of nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, antimetabolites, antibiotics, epipodophyllotoxins, platinum coordination complexes, vinca alkaloids, substituted ureas, methyl hydrazine derivatives, adrenocortical suppressants, endostatin, taxols, camptothecins, oxaliplatin, doxorubicin and doxorubicin analogs. Preferably, the chemotherapeutic agent is selected from the group consisting of doxorubicin, cisplatin, vincristine, dacarbazine (DTIC), cyclophosphamide, CPT-11, oxaliplatin, gemcitabine and 5-fluorouracil/leucovorin.

In any aspect, the cancer may be a blood born cancer or a solid tumour.

In any aspect, the cancer may be any one described herein, including one selected from the group consisting of brain cancer, oesophageal cancer, mouth cancer, tongue cancer, thyroid cancer, lung cancer, stomach cancer, pancreatic cancer, kidney cancer, colon cancer, rectal cancer, prostate cancer, bladder cancer, cervical cancer, epithelial cell cancers, skin cancer, neuroblastoma, leukaemia, lymphoma, myeloma, breast cancer, ovarian cancer, endometrial cancer and testicular cancer.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Chemotherapy treatment in myeloma RPMI-8226 and neuroblastoma Kelly cell lines. A) Normalised Ethidium influx in response to 0.5 mM BzATP stimulation in the Myeloma RPMI-8226 and Neuroblastoma Kelly cell lines. Mean of three independent experiments is shown. B) Effect of increasing doses of doxorubicin on RPMI-8226 cell viability measured using CellTitre-Blue (CTB) assay. C) Effect of increasing doses of doxorubicin on Kelly cell viability measured using CTB assay. D) Effect of increasing doses of 5 Fu on Kelly cell viability measured using CTB assay.

FIG. 2: Chemotherapy treatment in functional myeloma RPMI-8226 and non-functional neuroblastoma Kelly cell lines and induction of nfP2X7 as detected by BPM09. A) Effect of increasing amount of doxorubicin (0.0625 μM to 0.25 μM) on nfP2X7 antibody binding (here BPM09) to live RPMI-8226 cells by flow cytometry. B) Effect of increasing amount of doxorubicin (0.0625 μM to 0.25 μM) on nfP2X7 antibody binding (here BPM09) to live Kelly cells by flow cytometry. C) Effect of increasing amount of 5 Fu (1 μM to 8 μM) on nfP2X7 antibody binding to live Kelly cells by flow cytometry.

FIG. 3: Change in nfP2X7 antibody (here BPM09) binding to live ovarian A2780 parental cells and A2780 cells, which have acquired resistance to Doxorubicin and Cisplatin.

FIG. 4: BPM09 membrane scored after immunohistochemistry in a set of 200 patient derived xenograft models, which were previously treated or not with chemotherapy or radiotherapy. Similar analysis is performed in the colorectal dataset (37 samples).

FIG. 5: Effect of 5 Fu on nfP2X7 antibodies (here polyclonal mouse antibody) mediated complement dependent cytotoxicity on Kelly cells.

FIG. 6: A) Data showing that HMGB1 can drive the increase in nfP2X7. B) nfP2X7 induction in response to Condition Media from Doxorubicin treated cells is not blocked by P2X7 inhibitors. Condition Media from RPMI-8226 treated with Doxorubicin does not act via ATP

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

As used herein, except where the context requires otherwise, the term “comprise” and variations of the term, such as “comprising”, “comprises” and “comprised”, are not intended to exclude further additives, components, integers or steps.

All of the patents and publications referred to herein are incorporated by reference in their entirety.

For purposes of interpreting this specification, the following definitions will generally apply and whenever appropriate, terms used in the singular will also include the plural and vice versa. In the event that any definition set forth conflicts with any document incorporated herein by reference, the definition set forth below shall prevail.

The present inventors have surprisingly identified that chemotherapy or radiotherapy increases the level of P2X7 receptors that cannot form an apoptotic pore on live cells. Further, the inventors have identified that there is increased level of P2X7 receptors that cannot form an apoptotic pore on cells that have developed complete or partial resistance to chemotherapy. These findings are independent of the type of chemotherapy as chemotherapeutics of distinct structural classes that have distinct mechanisms of action, all result in increased level of P2X7 receptors that cannot form an apoptotic pore. Further, the findings apply to a wide variety of cancers irrespective of tissue origin (e.g. blood borne or solid). Lastly, the inventors have shown that this increased level driven by chemotherapy occurs in both cell lines and primary cells derived from patient samples.

The inventors have also shown that cancer cells pre-treated with chemotherapy, which therefore have an increased level of P2X7 receptors that cannot form an apoptotic pore, are sensitive to various interventions that target the P2X7 receptor.

Without being bound by any theory or mode of action, it surprisingly appears that the mechanism by which non-targeted therapies such as chemotherapy or radiotherapy increase the level of P2X7 receptors that cannot form an apoptotic pore on cells is not mediated via ATP. Instead, it appears that DAMPs such as HMGB1 are likely to mediate the effect.

### Definitions

“Purinergic receptor” generally refers to a receptor that uses a purine (such as ATP) as a ligand.

“P2X7 receptor” generally refers to a purinergic receptor formed from three protein subunits or monomers, with at least one, preferably all 3, of the monomers having an amino acid sequence substantially as shown in SEQ ID NO: 1. To the extent that P2X7 receptor is formed from three monomers, it is a “timer” or “trimeric”. “P2X7 receptor” may be a functional or non-functional receptor as described below. “P2X7 receptor” encompasses naturally occurring variants of P2X7 receptor, e.g., wherein the P2X7 monomers are splice variants, allelic variants and isoforms including naturally-occurring truncated or secreted forms of the monomers forming the P2X7 receptor (e.g., a form consisting of the extracellular domain sequence or truncated form of it), naturally-occurring variant forms (e.g., alternatively spliced forms) and naturally-occurring allelic variants. In certain embodiments of the invention, the native sequence P2X7 monomeric polypeptides disclosed herein are mature or full-length native sequence polypeptides comprising the full-length amino acids sequence shown in SEQ ID No:1. In certain embodiments the P2X7 receptor may have an amino acid sequence that is modified, for example various of the amino acids in the sequence shown in SEQ ID No:1 may be substituted, deleted, or a residue may be inserted.

“Functional P2X7 receptor” generally refers to a form of the P2X7 receptor having a binding site or cleft for binding to ATP. When bound to ATP, the receptor forms a non-selective cation channel that converts to a pore-like structure that enables the increased influx of calcium ions and also molecules of up to 1000 Da into the cytosol, one consequence of which may be programmed cell death. In normal homeostasis, expression of functional P2X7 receptors is generally limited to cells that undergo programmed cell death such as thymocytes, dendritic cells, lymphocytes, macrophages and monocytes. There may also be some expression of functional P2X7 receptors on erythrocytes and other cell types.

“Non-functional P2X7 receptor” or “nfP2X7” generally refers to a form of a P2X7 receptor having a conformation whereby the receptor is unable to form an apoptotic pore, but which is still able to operate as a non-selective channel. The isomerisation may arise from any molecular event that leads to misfolding of the monomer, including for example, mutation of monomer primary sequence or abnormal post translational processing. One consequence of the isomerisation is that the receptor is unable to extend the opening of the channel. In these circumstances, the receptor cannot form a pore and this limits the extent to which calcium ions and molecules of up to 1000 Da may enter the cytosol. Non-functional P2X7 receptors are expressed on a wide range of epithelial and haematopoietic cancers.

“Cancer associated—P2X7 receptors” are generally P2X7 receptors that are found on cancer cells (including, pre-neoplastic, neoplastic, malignant, benign or metastatic cells), but not on non-cancerous or normal cells.

“E200 epitope” generally refers to an epitope exposed on a non-functional P2X7 receptor. In humans the sequence is GHNYTTRNILPGLNITC (SEQ ID NO: 5).

“E300 epitope” generally refers to an epitope exposed on a non-functional P2X7 receptor. In humans the sequence is KYYKENNVEKRTLIKVF (SEQ ID NO: 8).

“Composite epitope” generally refers to an epitope that is formed from the juxtaposition of the E200 and E300 epitopes or parts of these epitopes.

As used herein, a “P2X7 receptor targeted therapy” is any therapy that directly or in-directly results in a reduction in the viability of a cancer cell expressing a P2X7 receptor has an impaired response to ATP such that it is unable to form an apoptotic pore under normal physiological conditions. Typically, the therapy comprises the administration of a molecule that binds to, induces an immune response to, or reduces the level of a P2X7 receptor has an impaired response to ATP such that it is unable to form an apoptotic pore under normal physiological conditions. Preferably the molecule that binds to a P2X7 receptor is an antibody or cell-based therapy. Preferably, the molecule that induces an immune response to P2X7 receptor is an immunogen in the individual in the form of a P2X7 receptor, or a fragment of a P2X7 receptor that is capable of inducing an immune response to a P2X7 receptor in the individual, wherein the P2X7 receptor has an impaired response to ATP such that it is unable to form an apoptotic pore under normal physiological conditions. Preferably, the molecule that reduces the level of P2X7 receptor is an interfering RNA. Inhibition of a P2X7 receptor that has an impaired response to ATP may also include a reduction in the level or amount of P2X7 receptor protein, RNA or DNA in a cell, preferably a cancer cell. The molecule may be specific for a P2X7 receptor and only have some low level inhibitory activity against other P2X receptors.

“Antibodies” or “immunoglobulins” or “Igs” are gamma globulin proteins that are found in blood, or other bodily fluids of vertebrates that function in the immune system to bind antigen, hence identifying and/or neutralizing foreign objects.

Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide bridges.

H and L chains define specific Ig domains. More particularly, each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1).

Antibodies can be assigned to different classes or isotypes. There are five classes of immunoglobulins: IgA, IgD, IgE, IgG, and IgM, having heavy chains designated α, δ, ε, γ, and μ, respectively. The γ and a classes are further divided into subclasses on the basis of relatively minor differences in C_{H} sequence and function, e.g., humans express the following subclasses: IgG1, IgG2, IgG3, IgG4, IgA1, and IgA2. The L chain from any vertebrate species can be assigned to one of two clearly distinct types, called kappa and lambda, based on the amino acid sequences of their constant domains.

The constant domain includes the Fc portion which comprises the carboxy-terminal portions of both H chains held together by disulfides. The effector functions of antibodies such as ADCC are determined by sequences in the Fc region, which region is also the part recognized by Fc receptors (FcR) found on certain types of cells.

The pairing of a V_{H} and V_{L} together forms a “variable region” or “variable domain” including the amino-terminal domains of the heavy or light chain of the antibody. The variable domain of the heavy chain may be referred to as “V_{H}.” The variable domain of the light chain may be referred to as “V_{L}.” The V domain contains an “antigen binding site” which affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each generally 9-12 amino acids long. The FRs largely adopt a β-sheet configuration and the hypervariable regions form loops connecting, and in some cases forming part of, the β-sheet structure.

“Hypervariable region” refers to the regions of an antibody variable domain which are hypervariable in sequence and/or form structurally defined loops. Generally, antibodies comprise six hypervariable regions; three in the V_{H} (HI, H2, H3), and three in the V_{L} (LI, L2, L3).

“Framework” or “FR” residues are those variable domain residues other than the hypervariable region residues herein defined.

An “antigen binding site” generally refers to a molecule that includes at least the hypervariable and framework regions that are required for imparting antigen binding function to a V domain. An antigen binding site may be in the form of an antibody or an antibody fragment, (such as a dAb, Fab, Fd, Fv, F(ab′)₂ or scFv) in a method described herein.

An “intact” or “whole” antibody is one which comprises an antigen-binding site as well as a C_{L} and at least heavy chain constant domains, C_{H}I, C_{H}2 and C_{H}3. The constant domains may be native sequence constant domains (e.g. human native sequence constant domains) or amino acid sequence variant thereof.

“whole antibody fragments including a variable domain” include Fab, Fab′, F(ab′)₂, and Fv fragments; diabodies; linear antibodies, single-chain antibody molecules; and multispecific antibodies formed from antibody fragments.

The “Fab fragment” consists of an entire L chain along with the variable region domain of the H chain (V_{H}), and the first constant domain of one heavy chain (C_{H}I). Each Fab fragment is monovalent with respect to antigen binding, i.e., it has a single antigen-binding site.

A “Fab′ fragment” differs from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group.

A “F(ab′)₂ fragment” roughly corresponds to two disulfide linked Fab fragments having divalent antigen-binding activity and is still capable of cross-linking antigen.

An “Fv” is the minimum antibody fragment which contains a complete antigen-recognition and binding site. This fragment consists of a dimer of one heavy and one light chain variable region domain in tight, non-covalent association.

In a single-chain Fv (scFv) species, one heavy and one light chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain FV” also abbreviated as “sFV” or “scFV” are antibody fragments that comprise the V_{H} and V_{L} antibody domains connected to form a single polypeptide chain. Preferably, the scFv polypeptide further comprises a polypeptide linker between the V_{H} and V_{L} domains which enables the scFv to form the desired structure for antigen binding.

A “single variable domain” is half of an Fv (comprising only three CDRs specific for an antigen) that has the ability to recognize and bind antigen, although at a lower affinity than the entire binding site.

“Diabodies” refers to antibody fragments with two antigen-binding sites, which fragments comprise a heavy-chain variable domain (V_{H}) connected to a light-chain variable domain (V_{L}) in the same polypeptide chain (V_{H}-V_{L}). The small antibody fragments are prepared by constructing sFv fragments (see preceding paragraph) with short linkers (about 5-10 residues) between the V_{H} and V_{L} domains such that interchain but not intra-chain pairing of the V domains is achieved, resulting in a bivalent fragment, i.e., fragment having two antigen-binding sites.

Diabodies may be bivalent or bispecific. Bispecific diabodies are heterodimers of two “crossover” sFv fragments in which the V_{H} and V_{L} domains of the two antibodies are present on different polypeptide chains. Triabodies and tetrabodies are also generally known in the art.

An “isolated antibody” is one that has been identified and separated and/or recovered from a component of its pre-existing environment. Contaminant components are materials that would interfere with therapeutic uses for the antibody, and may include enzymes, hormones, and other proteinaceous or nonproteinaceous solutes.

A “human antibody” refers to an antibody that possesses an amino acid sequence that corresponds to that of an antibody produced by a human. Human antibodies can be produced using various techniques known in the art, including phage-display libraries. Human antibodies can be prepared by administering the antigen to a transgenic animal that has been modified to produce such antibodies in response to antigenic challenge, but whose endogenous loci have been disabled.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesized uncontaminated by other antibodies. Monoclonal antibodies may be prepared by the hybridoma methodology. The “monoclonal antibodies” may also be isolated from phage antibody libraries using the techniques.

The term “anti-P2X7 receptor antibody” or “an antibody that binds to P2X7 receptor” refers to an antibody that is capable of binding P2X7 receptor with sufficient affinity such that the antibody is useful as a diagnostic and/or therapeutic agent in targeting P2X7 receptor, typically non-functional P2X7 receptor. Preferably, the extent of binding of an P2X7 receptor antibody to an unrelated protein is less than about 10% of the binding of the antibody to P2X7 receptor as measured, e.g., by a radioimmunoassay (RIA), Enzyme-Linked Immunosorbent Assay (ELISA), Biacore or Flow Cytometry. In certain embodiments, an antibody that binds to P2X7 receptor has a dissociation constant (Kd) of <1 uM, <100 nM, <10 nM, <1 nM, or <0.1 nM. An anti non-functional P2X7 receptor antibody is generally one having some or all of these serological characteristics and that binds to non-functional P2X7 receptors but not to functional P2X7 receptors.

An “affinity matured” antibody is one with one or more alterations in one or more hypervariable region thereof that result in an improvement in the affinity of the antibody for antigen, compared to a parent antibody that does not possess those alteration(s). Preferred affinity matured antibodies will have nanomolar or even picomolar affinities for the target antigen. Affinity matured antibodies are produced by procedures known in the art.

A “blocking” antibody” or an “antagonist” antibody is one that inhibits or reduces biological activity of the antigen it binds. Preferred blocking antibodies or antagonist antibodies substantially or completely inhibit the biological activity of the antigen.

An “agonist antibody”, as used herein, is an antibody, which mimics at least one of the functional activities of a polypeptide of interest.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

“Epitope” generally refers to that part of an antigen that is bound by the antigen binding site of an antibody. An epitope may be “linear” in the sense that the hypervariable loops of the antibody CDRs that form the antigen binding site bind to a sequence of amino acids as in a primary protein structure. In certain embodiments, the epitope is a “conformational epitope” i.e. one in which the hypervariable loops of the CDRs bind to residues as they are presented in the tertiary or quaternary protein structure.

‘Treatment’ generally refers to both therapeutic treatment and prophylactic or preventative measures.

Subjects requiring treatment include those already having a benign, pre-cancerous, or non-metastatic tumour as well as those in which the occurrence or recurrence of cancer is to be prevented.

The objective or outcome of treatment may be to reduce the number of cancer cells; reduce the primary tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the disorder.

Efficacy of treatment can be measured by assessing the duration of survival, time to disease progression, the response rates (RR), duration of response, and/or quality of life.

In one embodiment, the method is particularly useful for delaying disease progression.

In one embodiment, the method is particularly useful for extending survival of the human, including overall survival as well as progression free survival.

In one embodiment, the method is particularly useful for providing a complete response to therapy whereby all signs of cancer in response to treatment have disappeared. This does not always mean the cancer has been cured.

In one embodiment, the method is particularly useful for providing a partial response to therapy whereby there has been a decrease in the size of one or more tumours or lesions, or in the extent of cancer in the body, in response to treatment.

“Pre-cancerous” or “pre-neoplasia” generally refers to a condition or a growth that typically precedes or develops into a cancer. A “pre-cancerous” growth may have cells that are characterized by abnormal cell cycle regulation, proliferation, or differentiation, which can be determined by markers of cell cycle.

In one embodiment, the cancer is pre-cancerous or pre-neoplastic.

In one embodiment, the cancer is a secondary cancer or metastases. The secondary cancer may be located in any organ or tissue, and particularly those organs or tissues having relatively higher hemodynamic pressures, such as lung, liver, kidney, pancreas, bowel and brain.

Other examples of cancer include blastoma (including medulloblastoma and retinoblastoma), sarcoma (including liposarcoma and synovial cell sarcoma), neuroendocrine tumours (including carcinoid tumours, gastrinoma, and islet cell cancer), mesothelioma, schwannoma (including acoustic neuroma), meningioma, adenocarcinoma, melanoma, leukemia or lymphoid malignancies, lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumours of the biliary tract, as well as head and neck cancer.

“A condition or symptom associated” [with the cancer] may be any pathology that arises as a consequence of, preceding, or proceeding from the cancer. For example, where the cancer is a skin cancer, the condition or relevant symptom may be microbial infection. Where the cancer is a secondary tumour, the condition or symptom may relate to organ dysfunction of the relevant organ having tumour metastases. In one embodiment, the methods of treatment described herein are for the minimisation or treatment of a condition or symptom in an individual that is associated with a cancer in the individual.

A “non self” molecule, such as a “non self” antigen binding site, or “non self” antibody generally refers to a molecule that has been produced outside of, or exogenous to, a body in which the molecule is to be provided, for example, for treatment. As an example, synthetic or recombinant molecules are “non self”. Further, a molecule that is generated in one individual and administered to another individual for treatment is “non self”. “Non self” antigen binding sites and antibodies may be used in accordance with the invention for adoptive transfer of immunity, for example, as occurs in antibody infusion. In contrast, a molecule that is generated inside an individual that is to be treated with that molecule, is generally a “self” or “endogenous” molecule. One example of a “self” molecule is an antigen binding site or antibody that is generated, or arises from an adaptive immune response to immunogen.

“level of non self-antigen binding sites in circulation” in the individual generally refers to the concentration of antigen binding site in a body fluid, preferably peripheral blood.

A “substantially undetectable level of non self-antigen binding sites in circulation” generally refers to a concentration of exogenous antigen binding sites (i.e. those that have been administered by adoptive transfer) that is at least half of the concentration of the antigen binding sites in circulation at the time of administration of the antigen binding sites, preferably 25%, or 10%, or 5% or 1% of said concentration, or otherwise less than 0.001 mg/kg of the individual. The phrase may also refer to a circumstance where antigen binding sites that have been given for the purpose of cancer immunotherapy cannot be detected at all.

A cancer that is “substantially undetectable” generally refers to a circumstance where therapy has depleted the size, volume or other physical measure of a cancer so that using relevant standard detection techniques such as in vivo imaging, the cancer, as a consequence of the therapy, is not clearly detectable. The phrase also refers to the circumstance where the cancer cannot be detected at all.

“forming an immune response” generally refers to invoking or inducing antigen specific immunity via the adaptive immune system. As is generally understood in the art, induction of antigen specific immunity is distinguished from adoptive transfer of immunity, standard cancer immunotherapy by administration of exogenous or non self-antibody being one example of the latter.

Individuals Selected for Treatment

In one aspect, the individuals selected for treatment according to a method described above are those who have received, or who are continuing to receive chemotherapy, for treatment of cancer. For example, the individual may have received any one or more of the chemotherapies described herein including oxazaphosphorines, topoisomerase I inhibitors, topoisomerase II inhibitors, proteasome inhibitors, antifolates, nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, purine antagonists, antimetabolites, antibiotics, epipodophyllotoxins, platinum based agents, ribonucleotide reductase inhibitors, vinca alkaloids, substituted ureas, hydrazine derivatives, adrenocortical suppressants, endostatin, camptothecins, oxaliplatin, doxorubicin and doxorubicin analogs, antibiotics, L-asparaginase, tyrosine kinase inhibitors, or derivatives or variants thereof.

In one embodiment, the individual may have received chemotherapy leading to a reduced tumour mass but one that is still clinically or biochemically detectable. For example, at the time the P2X7 targeted treatment is applied the cancer may have substantially diminished in size, mass or other physical measure as a consequence of chemotherapy.

Further, the individual selected for treatment according to a method described above may or may not have detectable cancer at the time of treatment.

In another aspect, an individual selected for treatment is one who has not responded, or no longer responds, to chemotherapy.

The purpose of the treatment according to the above described methods is to at least minimise the progression of cancer. One approach to treatment is by induction or formation of an immune response in the individual to a non-functional P2X7 receptor.

Therefore, the individual selected for this form of treatment must be capable of generating an immune response sufficient for meeting this purpose. Generally, the desired immune response includes a capacity to produce either or both of circulating IgM and IgG when the individual is challenged by cancer, as in recurrence of cancer.

Antigen Binding Sites and Administration

One approach to P2X7 receptor targeted therapy is administration of antigen binding sites, or antibodies, that bind to a P2X7 receptor that has an impaired response to ATP such that it is unable to form an apoptotic pore under normal physiological conditions.

Typically, the antigen binding site is one that discriminates between functional and non-functional P2X7 receptors, so as to bind to non-functional receptors, but not to functional receptors. Examples of these antigen binding sites are those that bind to the E200 epitope, E300 epitope or composite epitope as for example in PCT/AU2002/000061 and PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), in PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or 8,658,385), PCT/AU2009/000869 (or in any one of the corresponding U.S. Pat. Nos. 8,597,643, 9,328,155 or 10,238,716) and PCT/AU2010/001070 (or in any one of the corresponding U.S. Pat. Nos. 9,127,059, 9,688,771, or U.S. Pat. No. 10,053,508), all of which are hereby incorporated by reference.

In certain embodiments the antigen binding cite comprises the CDR amino acid sequences of 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

Regardless of specificity, (i.e. P2X7 receptor specific or otherwise), the antigen binding site may take the form of a whole antibody, or a whole antibody fragment such as a Fab, a Fab′, a F(ab′)₂, and Fv, a single chain Fv, or a single variable domain.

The antigen binding site may be syngeneic, allogeneic or xenogeneic.

Typically, the antigen binding site is non self or exogenous meaning that it has been found or isolated outside of the individual who is treated according to the methods of the invention.

The antigen binding site may be affinity matured.

The antigen binding site may have multiple specificities or valencies.

The antigen binding site may be adapted so as to be suited to administration by a selected method.

The antibody may be a whole antibody of any isotype. The antibody may be one obtained from monoclonal or polyclonal antisera. The antibody may be produced by hybridoma, or by recombinant expression, or may be obtained from serum for example as obtainable from a mammal, particularly a human or mouse. The antibody may also be obtained from an avian.

The antibody may be chimeric, i.e. one containing human variable domains and non-human constant domains. Alternatively, it may be humanized, i.e. one formed by grafting non-human CDRs onto a human antibody framework. Still further, the antibody may be fully human.

The antibody may be modified with respect to effector function, so as to enhance, e.g., the effectiveness of the antibody in treating cancer.

Where the antibody is an antibody fragment, the antibody fragment is selected from the group consisting of a dAb, Fab, Fd, Fv, F(ab′)₂, scFv and CDR.

Dosage amount, dosage frequency, routes of administration etc are described in detail below.

Methods of preparing and administering antibodies to a subject in need thereof are well known to, or are readily determined by those skilled in the art. The route of administration may be, for example, oral, parenteral (e.g. intravenous, intraarterial, intraperitoneal, intramuscular, subcutaneous, intradermal, rectal or vaginal), by inhalation or topical. One form for administration would be a solution for injection, in particular for intravenous or intraarterial injection or drip, comprising a buffer (e.g. acetate, phosphate or citrate buffer), a surfactant (e.g. polysorbate), optionally a stabilizer agent (e.g. human albumin). In other methods antibodies can be delivered directly to the site of disease thereby increasing the exposure of the diseased cell or tissue to the antibody.

Preparations for parenteral administration includes sterile aqueous (aqueous carriers include water, alcoholic/aqueous solutions, emulsions or suspensions, including saline and buffered media) or non-aqueous (non-aqueous solvents are propylene glycol, polyethylene glycol, vegetable oils such as olive oil, and injectable organic esters such as ethyl oleate) solutions, suspensions, and emulsions. Pharmaceutically acceptable carriers include 0.01-0.1M and preferably 0.05M phosphate buffer or 0.9% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions, in such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminium monostearate and gelatin.

In any case, sterile injectable solutions can be prepared by incorporating an active compound (e.g., antigen binding site) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying, freeze-drying and spray drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit. Such articles of manufacture will preferably have labels or package inserts indicating that the associated compositions are useful for treating a subject suffering from, or predisposed to disorders.

Effective doses of the compositions of the present invention, for treatment of disorders, as described herein, vary depending upon many different factors, including means of administration, target site, physiological state of the patient, whether the patient is human or an animal, other medications administered, and whether treatment is prophylactic or therapeutic. Treatment dosages may be titrated using routine methods known to those of skill in the art to optimize safety and efficacy.

For treatment of certain disorders with an antibody, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimens entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more antigen binding sites with different binding specificities are administered simultaneously, in which case the dosage of each antigen binding site administered falls within the ranges indicated.

The antibody for binding to a non-functional P2X7 receptor expressed on a cell can be administered on multiple occasions. Intervals between single dosages can be weekly, monthly or yearly. Intervals can also be irregular as indicated by measuring blood levels of target polypeptide or target molecule in the patient. In some methods, dosage is adjusted to achieve a plasma polypeptide concentration of 1-1000 ug/mL and in some methods 25-300 ug/mL. Alternatively, the antibody can be administered as a sustained release formulation, in which case less frequent administration is required. Dosage and frequency vary depending on the half-life of the antibody in the patient. The half-life of an antibody can also be prolonged via fusion to a stable polypeptide or moiety, e.g., albumin or PEG. In general, humanized antibodies show the longest half-life, followed by chimeric antibodies and nonhuman antibodies. In one embodiment, the antibody can be administered in unconjugated form. In another embodiment the antibody can be administered multiple times in conjugated form. In certain therapeutic applications, a relatively high dosage (e.g., up to 400 mg/kg of anti P2X7 binding molecule, e.g., antibody per dose), at relatively short intervals is sometimes required until progression of the disease is reduced or terminated, and preferably until the patient shows partial or complete amelioration of symptoms of disease. The amounts can be several logs lower (i.e. 2 to 3 logs lower) where the antibody is conjugated to a radioisotope or cytotoxic drug.

Therapeutic agents can be administered by parenteral, topical, intravenous, oral, subcutaneous, intraarterial, intracranial, intraperitoneal, intranasal or intramuscular means for prophylactic and/or therapeutic treatment, in some methods, agents are injected directly into a particular tissue where non-functional P2X7 receptor cells have accumulated, for example intracranial injection. Intramuscular injection or intravenous infusion are preferred for administration of antibody.

An antibody can optionally be administered in combination with other agents that are effective in treating the disorder or condition in need of treatment (e.g., prophylactic or therapeutic). Examples are agents commonly used for chemotherapy or radiotherapy in oncology. Additionally or alternatively, the antibody or agent may be administered before, during or after surgical intervention for resection or removal of tumour or tissue.

Immunogens and Forming an Immune Response

Another P2X7 receptor targeted therapy is the formation of an immune response in an individual to be treated to a P2X7 receptor, especially a non-functional P2X7 receptor. Generally the immunogen, which is used for the purpose, is one which elicits an immune response to non-functional P2X7 but not to functional P2X7 receptors.

The immunogen may include or consist of a peptide including a sequence of a P2X7 receptor. The peptide may contain at least one sequence that is capable of being presented on a major histocompatibility complex class II molecule or, that is capable of interacting with a B-cell receptor or a B-cell membrane bound-immunoglobulin. Typically the peptide includes a sequence of a human P2X7 receptor or fragment thereof.

A range of peptide immunogens are known and discussed in PCT/AU2002/000061, or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2008/001364 (or in any one of the corresponding U.S. Pat. Nos. 8,440,186, 9,181,320, 9,944,701 or U.S. Pat. No. 10,597,451), and PCT/AU2009/000869 (or in any one of the corresponding U.S. Pat. Nos. 8,597,643, 9,328,155 or 10,238,716), the contents of which are incorporated in entirety.

Exemplary peptide immunogens within these specifications, which include epitopes for generating an immune response to a non-functional P2X7 receptor are described below.

| | Peptide immunogen |
|---|---|
| PCT application | sequence |
| PCT/AU2002/000061, | GHNYTTRNILPGLNITC |
| PCT/AU2002/001204 | (SEQ ID NO: 5) |
| (U.S. Pat. No. 7,326,415, | |
| U.S. Pat. No. 7,888,473, | |
| U.S. Pat. No. 7,531,171, | |
| U.S. Pat. No. 8,080,635, | |
| U.S. Pat. No. 8,399,617, | |
| U.S. Pat. No. 8,709,425, | |
| U.S. Pat. No. 9,663,584, | |
| or U.S. Pat. No. 10,450,380) | |
| PCT/AU2008/001364 | KYYKENNVEKRTLIKVF |
| (U.S. Pat. No. 8,440,186, | (SEQ ID NO: 8) |
| U.S. Pat. No. 9,181,320, | |
| U.S. Pat. No. 9,944,701 | |
| or U.S. Pat. No. 10,597,451) | |
| PCT/AU2009/000869 | GHNYTTRNILPGAGAKY |
| (U.S. Pat. No. 8,597,643, | YKENNVEK (SEQ ID |
| U.S. Pat. No. 9,328,155 | NO: 11) |
| or U.S. Pat. No. 10,238,716) | |

It will be understood that these are merely examples of possible immunogens useful for forming an immune response according to the methods of the invention described herein. Further, the invention includes the use of other peptides as described in these applications useful for forming an immune response to non-functional P2X7 receptors.

Typically, the immunisation regime involves 2 or more immunisations. In a first immunisation, the objective may be to develop an IgM response to immunisation. A second immunisation may be to develop an IgG response. Further immunisations may be to boost the IgG response, as discussed further below.

Where the immunogen is a peptide, the peptide may be provided in an amount of about 0.1 to 2 mg per administration, preferably about 0.25 to 1 mg, preferably about 0.5 mg.

A further administration of about 0.25 to 1 mg peptide may be applied as a boost.

In one embodiment, a first immunisation is performed when the circulating level of antigen binding sites that had been administered for antibody immunotherapy is substantially undetectable. In other words, circulating antibody to the relevant cancer biomarker cannot be detected in peripheral blood. The level of IgM production is then monitored over the following weeks. At about 4 to 5 weeks after first immunisation, the level of IgM antibody is likely to have decreased to negligible circulating levels. At this point, a second immunisation is then performed and the level of IgG production is monitored over the following weeks.

After boosting, the level of antibody produced may be 0.1-25 mg/kg, for example from 0.1 to 10 mg/kg, preferably 5 mg/kg, or from 10 to 25 mg/kg, preferably 15 mg/kg and more than 10 mg/kg. Whether this amount is detected in circulation will depend on whether there is existing tumour mass. Where there is existing tumour mass capable of binding to antibody formed by the humoral response, the level of antibody detected in circulation may be at the lower end of this range, or indeed outside the lower end of the range (i.e. less than 0.1 mg/kg), or otherwise substantially undetectable. Where there is no detectable tumour mass, the level of antibody formed from the humoral response may be at the higher end of this range, although in certain embodiments, in these circumstances, an amount of about 5 mg/kg antibody may be sufficient. Further testing of immunity over the following months/years may be performed and boosting immunisations may be provided as required.

The degree or number of boosts may depend on the patient status and response. Where scans or lack of free circulating antibody are indicative of extant tumour burden, then boosts may be performed monthly, ideally to ensure sufficient reaction of the immune system. Where the free levels of antibody in serum rise, the boosts may then be eased off and perhaps applied 6-12 monthly subject to clinical observation.

As discussed above, the immune response may target a biomarker that is different to the biomarker that has been targeted by antibody immunotherapy. For example, anti CD20 antibody may be used for antibody immunotherapy and a non-functional P2X7 immunogen used for generating an immune response.

In another embodiment, a single biomarker is targeted by antibody immunotherapy and immunisation. For example, a monoclonal antibody directed to one epitope on a P2X7 receptor (such as the E300 epitope) may be used for antibody immunotherapy, and an immunogen for forming an immune response that targets a different epitope (such as the E200 epitope) on P2X7 may be used for immunisation.

A peptide immunogen for use in a method of the invention herein may have a length of 6, 7, 8, 9, 10, 11, 12, 13, 14, 15, 16, 17, 18, 19, 20, 21, 22, 23, 24, 25 or 26 residues.

In one embodiment, the immunogen for forming an immune response according to a method of the invention is a peptide having a sequence of a P2X7 receptor that may or may not have Pro210 in cis conformation.

The immunogen may be in the form of a P2X7 extracellular domain or any one or more of the P2X7 isoforms. The immunogen may be provided for administration in a soluble form or associated with a solid phase such as a cell membrane, bead, or other surface.

Methods for screening peptides that can be used as an immunogen to form an immune response according to the methods of the invention herein are disclosed herein. One example includes the use of erythrocytes in a rosetting assay. In this assay an antibody that binds to functional receptors is used as a positive control in which rossettes are observed. A test antibody is determined not to bind to functional receptors if it fails to form rossettes. It is determined to bind to non-functional receptors if it is observed to bind to a non-functional receptor-expressing cell line, including those discussed herein.

The peptides of the invention can be made by any number of techniques known in the art including solid phase synthesis and recombinant DNA technology.

As is known in the art, a carrier is a substance that may be conjugated to a peptide epitope thereby enhancing immunogenicity. Some carriers do this by binding to multiple peptides so as to provide an antigen of increased molecular weight to the host in which the immune response is to be developed.

Preferred carriers include bacterial toxins or toxoids. Other suitable carriers include the N. meningitides outer membrane protein, albumin such as bovine serum albumin, synthetic peptides, heat shock proteins, KLH, Pertussis proteins, protein D from H. influenza and toxin A, B or C from C. difficile.

When the carrier is a bacterial toxin or toxoid, diphtheria or tetanus toxoids are preferred.

Preferably the carrier contains functional groups that can react with the peptide of the invention, or may be modified to be capable of reacting with the peptide.

The immunogen may be administered subcutaneously, intradermally and/or intramuscularly.

Adjuvants

In a preferred form, the composition for forming an immune response to a P2X7 receptor for use in the methods of the invention described herein includes an adjuvant or compound for potentiating an immune response.

A large number of adjuvants are known; See also Allison (1998, Dev. Biol. Stand., 92:3-11; incorporated herein by reference), Unkeless et al. (1998, Annu. Rev. Immunol., 6:251-281), and Phillips et al. (1992, Vaccine, 10:151-158). Exemplary adjuvants that can be utilized in accordance with the invention include, but are not limited to, cytokines, aluminium salts (e.g., aluminium hydroxide, aluminium phosphate, etc.; Baylor et al., Vaccine, 20:S18, 2002), gel-type adjuvants (e.g., calcium phosphate, etc.); microbial adjuvants (e.g., immunomodulatory DNA sequences that include CpG motifs; endotoxins such as monophosphoryl lipid A (Ribi et al., 1986, Immunology and Immunopharmacology of bacterial endotoxins, Plenum Publ. Corp., N.Y., p407, 1986); exotoxins such as cholera toxin, E. coli heat labile toxin, and pertussis toxin; muramyl dipeptide, etc.); oil-emulsion and emulsifier-based adjuvants (e.g., Freund's Adjuvant, MF59 [Novartis], SAF, etc.); particulate adjuvants (e.g., liposomes, biodegradable microspheres, etc.); synthetic adjuvants (e.g., nonionic block copolymers, muramyl peptide analogues, polyphosphazene, synthetic polynucleotides, etc.); and/or combinations thereof. Other exemplary adjuvants include some polymers (e.g., polyphosphazenes; described in U.S. Pat. No. 5,500,161), Q57, saponins (e.g., QS21, Ghochikyan et al., Vaccine, 24:2275, 2006), squalene, tetrachlorodecaoxide, CPG 7909 (Cooper et al., Vaccine, 22:3136, 2004), poly[di(carboxylatophenoxy)phosphazene] (PCCP; Payne et al., Vaccine, 16:92, 1998), interferon-γ (Cao et al., Vaccine, 10:238, 1992), block copolymer P1205 (CRL1005; Katz et al., Vaccine., 18:2177, 2000), interleukin-2 (IL-2; Mbwuike et al., Vaccine, 8:347, 1990), polymethyl methacrylate (PMMA; Kreuter et al., J. Pharm. ScL, 70:367, 1981), etc.

In one embodiment, a peptide immunogen containing a sequence of a P2X7 receptor is provided on the surface of a bacteriophage for immunisation of an individual according to a method of the invention described herein.

Cell Based Therapy

Another P2X7 receptor targeted therapy includes a cell-based therapy. Specifically, the present invention provides a method of treating cancer in an individual who has not responded, or no longer responds, to chemotherapy, the method comprising
providing an individual who has not responded, or no longer responds, to a chemotherapeutic agent;
providing in the individual a cell-based therapy that targets P2X7 receptor expressing cancer cells;
wherein the P2X7 receptor has an impaired response to ATP such that it is unable to form an apoptotic pore under normal physiological conditions,

thereby treating cancer in the individual.

In any aspect, a cell-based therapy that targets P2X7 receptor expressing cancer cells may be a CAR-T cell or other cytotoxic cell that has the capacity to bind to a P2X7 receptor expressing cancer cell.

Chimeric antigen receptor T cells (CAR-T cells) are T cells that have been genetically engineered to produce an artificial T-cell receptor (a chimeric antigen receptor). A chimeric antigen receptor useful in the present invention includes an antigen-recognition domain and a signalling domain, wherein the antigen-recognition domain recognises a P2X7 receptor has an impaired response to ATP such that it is unable to form an apoptotic pore under normal physiological conditions.

A chimeric antigen receptor (CAR) comprises an extracellular and intracellular domain. The extracellular domain comprises a target-specific binding element otherwise referred to as an antigen binding moiety. The intracellular domain or otherwise the cytoplasmic domain comprises, a costimulatory signalling region and a zeta chain portion. The costimulatory signalling region refers to a portion of the CAR comprising the intracellular domain of a costimulatory molecule. Costimulatory molecules are cell surface molecules other than antigens receptors or their ligands that are required for an efficient response of lymphocytes to antigen.

Between the extracellular domain and the transmembrane domain of the CAR, or between the cytoplasmic domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 600 amino acids, preferably 10 to 100 amino acids and most preferably 25 to 50 amino acids. The spacer domain may consist of entire antibody Fc domains and additionally possess appropriate linkers that may separate one or more binding domains.

Antigen Recognition Domain

The CAR useful in the present invention comprises a target-specific binding element otherwise referred to as an antigen recognition domain or antigen binding moiety that binds to a P2X7 receptor. Preferably, the P2X7 receptor has an impaired response to ATP such that it is unable to form an apoptotic pore under normal physiological conditions.

Suitable antigen recognition domains or antigen binding moieties are described herein and include any antigen binding site or antigen binding domain described herein in single or multiple formats separated appropriately for the purpose of conformational optimisation.

Transmembrane Domain

With respect to the transmembrane domain, the CAR can be designed to comprise a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CDS, CD9, CD16, CD22, CD33, CD37, CD64, CD80, CD86, CD 134, CD137, CD 154. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

Optionally, a short oligo- or polypeptide linker, preferably between 2 and 10 amino acids in length may form the linkage between the transmembrane domain and the cytoplasmic signalling domain of the CAR. A glycine-serine doublet provides a particularly suitable linker.

Preferably, the transmembrane domain in the CAR of the invention is the CD8 transmembrane domain.

In some instances, the transmembrane domain of the CAR of the invention comprises the CD8a hinge domain.

Cytoplasmic Domain

The cytoplasmic domain or otherwise the intracellular signalling domain of the CAR of the invention is responsible for activation of at least one of the normal effector functions of the immune cell in which the CAR has been placed. The term “effector function” refers to a specialized function of a cell. Effector function of a T cell, for example, may be cytolytic activity or helper activity including the secretion of cytokines. Thus the term “intracellular signalling domain” refers to the portion of a protein, which transduces the effector function signal and directs the cell to perform a specialized function. While usually the entire intracellular signalling domain can be employed, in many cases it is not necessary to use the entire chain. To the extent that a truncated portion of the intracellular signalling domain is used, such truncated portion may be used in place of the intact chain as long as it transduces the effector function signal. The term intracellular signalling domain is thus meant to include any truncated portion of the intracellular signalling domain sufficient to transduce the effector function signal.

Preferred examples of intracellular signalling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signalling sequence: those that initiate antigen-dependent primary activation through the TCR (primary cytoplasmic signalling sequences) and those that act in an antigen-independent manner to provide a secondary or co-stimulatory signal (secondary cytoplasmic signalling sequences).

Primary cytoplasmic signalling sequences regulate primary activation of the TCR complex either in a stimulatory way, or in an inhibitory way. Primary cytoplasmic signalling sequences that act in a stimulatory manner may contain signalling motifs which are known as immunoreceptor tyrosine-based activation motifs or ITAMs.

Examples of ITAM containing primary cytoplasmic signalling sequences that are of particular use in the invention include those derived from TCR zeta, FcR gamma, FcR beta, CD3 gamma, CD3 delta, CD3 epsilon, CDS, CD22, CD79a, CD79b, and CD66d. It is particularly preferred that cytoplasmic signalling molecule in the CAR of the invention comprises a cytoplasmic signalling sequence derived from CD3 zeta.

In a preferred embodiment, the cytoplasmic domain of the CAR can be designed to comprise the CD3-zeta signalling domain by itself or combined with any other desired cytoplasmic domain(s) useful in the context of the CAR of the invention. For example, the cytoplasmic domain of the CAR can comprise a CD3 zeta chain portion and a costimulatory signalling region. The costimulatory signalling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-IBB (CD 137), 0X40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Thus, while the invention in exemplified primarily with 4-1BB as the co-stimulatory signalling element, other costimulatory elements are within the scope of the invention.

The cytoplasmic signalling sequences within the cytoplasmic signalling portion of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain is designed to comprise the signalling domain of CD3-zeta and the signalling domain of CD28. In another embodiment, the cytoplasmic domain is designed to comprise the signalling domain of CD3-zeta and the signalling domain of 4-IBB. In yet another embodiment, the cytoplasmic domain is designed to comprise the signalling domain of CD3-zeta and the signalling domain of CD28 and 4-1BB. In one embodiment, the cytoplasmic domain in the CAR of the invention is designed to comprise the signalling domain of 4-1BB and the signalling domain of CD3-zeta.

CAR-T cells useful in the present invention includes those described in PCT/AU2016/050851 (or in corresponding US publication 2019-0365805) the entire contents of which are incorporated by reference in their entirety.

Interfering RNA and Administration

One approach to treatment is administration of interfering RNA that reduce the level of P2X7 receptor on the surface of a cancer cell. The interfering RNA per se does not need to be specific for a P2X7 receptor that has an impaired response to ATP such that it is unable to form an apoptotic pore under normal physiological conditions. Instead, the interfering RNA may reduce total P2X7 receptor levels in a cell, and be targeted to a cell expressing P2X7 receptor that has an impaired response to ATP such that it is unable to form an apoptotic pore under normal physiological conditions. In other words, the interfering RNA may be targeted to cancer cells using any method known in the art.

Exemplary interfering RNA molecules are described in Gilbert et al. Oncogene. 2019 January; 38(2):194-208, the entire contents of which are incorporated by reference.

Exemplary siRNA target sequences include siRNA

| | A: |
|---|---|
| (SEQ ID NO: 13) | |
| | 5′-CCCGCAGAGCAAAGGAATTCAGACC-3′ |
| | B: |
| (SEQ ID NO: 12) | |
| | 5′-GAGATATTGTGAGGACAAATTGAGA-3′ |

Cancers and Conditions Associated Therewith

Pre-neoplastic, neoplastic and metastatic diseases are particular examples to which the methods of the invention may be applied. Broad examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilm's tumour.

Examples of particular cancers include but are not limited to adenocarcinoma, adenoma, adenofibroma, adenolymphoma, adontoma, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, branchioma, CNS tumours, carcinoid tumours, cervical cancer, childhood brain tumours, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumour, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumour, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumour, genitourinary cancers, germ cell tumours, gestationaltrophoblastic-disease, glioma, gynaecological cancers, giant cell tumours, ganglioneuroma, glioma, glomangioma, granulosa cell tumour, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. b-cell, mixed cell, null-cell, t-cell, t-cell chronic, htiv-ii-associated, lymphangiosarcoma, lymphocytic acute, lymphocytic chronic, mast-cell and myeloid), leukosarcoma, leydig cell tumour, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumours, squamous-cell-carcinoma-(skin), stomach cancer, synovial sarcoma, sarcoma (e.g. Ewing's experimental, Kaposi's and mast-cell sarcomas), Sertoli cell tumour, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumour, thymoma, trophoblastic tumour, urethral cancer, urinary system cancer, uroplakins, uterine sarcoma, uterus cancer, vaginal cancer, vulva cancer, Waldenstrom's-macroglobulinemia and Wilms' tumour.

The cancer may be one selected from the group consisting of brain cancer, oesophageal cancer, mouth cancer, tongue cancer, thyroid cancer, lung cancer, stomach cancer, pancreatic cancer, kidney cancer, colon cancer, rectal cancer, prostate cancer, bladder cancer, cervical cancer, epithelial cell cancers, skin cancer, leukaemia, lymphoma, myeloma, breast cancer, ovarian cancer, endometrial cancer and testicular cancer.

The cancer may be one selected from the group consisting of; lung cancer, oesophageal cancer, stomach cancer, colon cancer, prostate cancer, bladder cancer, cervical cancer, vaginal cancers, epithelial cell cancers, skin cancer, blood-related cancers, breast cancer, endometrial cancer, uterine cancer, cervix, cancer and testicular cancer.

The cancer may be metastatic.

The cancer may be stage III cancer.

The cancer may be stage IV cancer.

Kits

In another embodiment there is provided a kit or article of manufacture including:
an antigen binding site in the form of an immunoglobulin variable domain, antibody, dAb, Fab, Fd, Fv, F(ab′)₂, scFv or CDR reactive with a P2X7 receptor, preferably a non-functional P2X7 receptor;
an immunogen for generating an immune response to a non-functional P2X7 receptor; and
a label or package insert with instructions for use in a method described herein.

Amino Acid Sequences

Exemplary amino acid sequences as described herein are recited below:

(SEQ ID NO: 1)

| | |
|---|---|
| 1 | MPACCSCSDV FQYETNKVTR IQSMNYGTIK WFFHVIIFSY |
| | VCFALVSDKL YQRKEPVISS |
| 61 | VHTKVKGIAE VKEEIVENGV KKLVHSVFDT ADYTFPLQGN |
| | SFFVMTNFLK TEGQEQRLCP |
| 121 | EYPTRRTLCS SDRGCKKGWM DPQSKGIQTG RCVVHEGNQK |
| | TCEVSAWCPI EAVEEAPRPA |
| 181 | LLNSAENFTV LIKNNIDFPG HNYTTRNILP GLNITCTFHK |
| | TQNPQCPIFR LGDIFRETGD |
| 241 | NFSDVAIQGG IMGIEIYWDC NLDRWFHHCR PKYSFRRLDD |
| | KTTNVSLYPG YNFRYAKYYK |
| 301 | ENNVEKRTLI KVFGIRFDIL VFGTGGKFDI IQLVVYIGST |
| | LSYFGLAAVF IDFLIDTYSS |
| 361 | NCCRSHIYPW CKCCQPCVVN EYYYRKKCES IVEPKPTLKY |
| | VSFVDESHIR MVNQQLLGRS |
| 421 | LQDVKGQEVP RPAMDFTDLS RLPLALHDTP PIPGQPEEIQ |
| | LLRKEATPRS RDSPVWCQCG |
| 481 | SCLPSQLPES HRCLEELCCR KKPGACITTS ELFRKLVLSR |
| | HVLQFLLLYQ EPLLALDVDS |
| 541 | TNSRLRHCAY RCYATWRFGS QDMADFAILP SCCRWRIRKE |
| | FPKSEGQYSG FKSPY |

(SEQ ID NO: 2)
HNYTTRNIL
(SEQ ID NO: 3)
GHNYTTRNIL
(SEQ ID NO: 4)
DFPGHNYTTRNIL
(SEQ ID NO: 5)
GHNYTTRNILPGLNITC
(SEQ ID NO: 6)

| | |
|---|---|
| 1 | MPACCSCSDV FQYETNKVTR IQSMNYGTIK WFFHVIIFSY |
| | VCFALVSDKL YQRKEPVISS |
| 61 | VHTKVKGIAE VKEEIVENGV KKLVHSVFDT ADYTFPLQGN |
| | SFFVMTNFLK TEGQEQRLCP |
| 121 | EYPTRRTLCS SDRGCKKGWM DPQSKGIQTG RCVVHEGNQK |
| | TCEVSAWCPI EAVEEAPRPA |
| 181 | LLNSAENFTV LIKNNIDFPG HNYTTRNIL |

(SEQ ID NO: 7)
KTTNVSLYPGYNFRYAKYYKENNVEKRTLIKVFGIRFDILVFGTGGKFD
(SEQ ID NO: 8)
KYYKENNVEKRTLIKVF
(SEQ ID NO: 9)
GHNYTTRNILP
(SEQ ID NO: 10)
AKYYKENNVEK
(SEQ ID NO: 11)
GHNYTTRNILPGAGAKYYKENNVEK.

It will be understood that the invention disclosed and defined in this specification extends to all alternative combinations of two or more of the individual features mentioned or evident from the text or drawings. All of these different combinations constitute various alternative aspects of the invention.

### EXAMPLES

The experimental data described below has identified that chemotherapy or radiotherapy increases the level of P2X7 receptors that cannot form an apoptotic pore on live cells. Further, the data shows an increased level of P2X7 receptors that cannot form an apoptotic pore on cells that have developed complete or partial resistance to chemotherapy. These findings are independent of the type of chemotherapy as chemotherapeutics of distinct structural classes that have distinct mechanisms of action, all result in increased level of P2X7 receptors that cannot form an apoptotic pore. Further, the findings apply to a wide variety of cancers irrespective of tissue origin (e.g. blood borne or solid). Lastly, the experimental data shows that this increased level driven by chemotherapy occurs in both cell lines and primary cells derived from patient samples. The cancer cells pre-treated with chemotherapy, which therefore have an increased level of P2X7 receptors that cannot form an apoptotic pore, are sensitive to various interventions that target the P2X7 receptor.

### Example 1

Chemotherapy Treatment in Myeloma RPMI-8226 and Neuroblastoma Kelly Cell Lines.

Materials and Methods

4,000 cells were seeded in a 96 well plate and allowed to adhere over-night. Cells were then treated with increasing amount of chemotherapy or vehicle control for 72 hours to induce varying amount of cell killing. Cell viability was then measured using CellTiter-Blue Cell Viability Assay form Promega following manufacturer's instruction.

Normalised ethidium influx in response to 0.5 mM BzATP stimulation in the myeloma RPMI-8226 and neuroblastoma Kelly cell lines. Mean of three independent experiments is shown.

Results

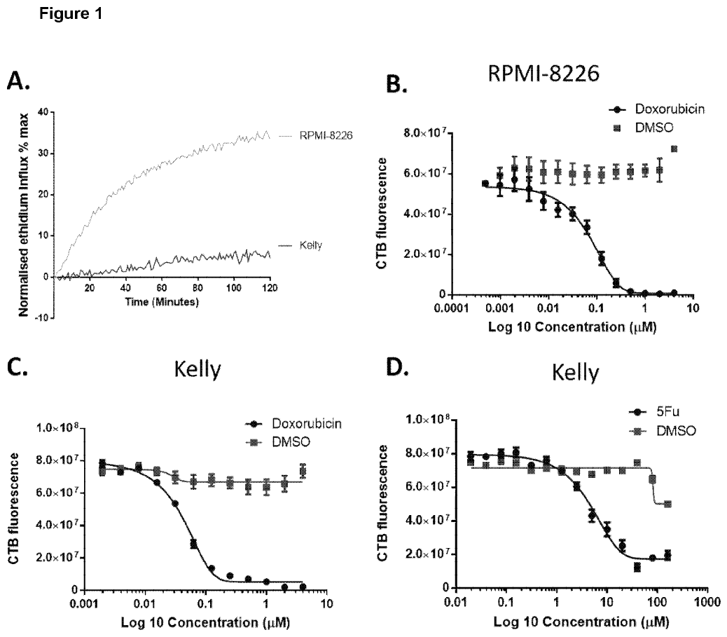

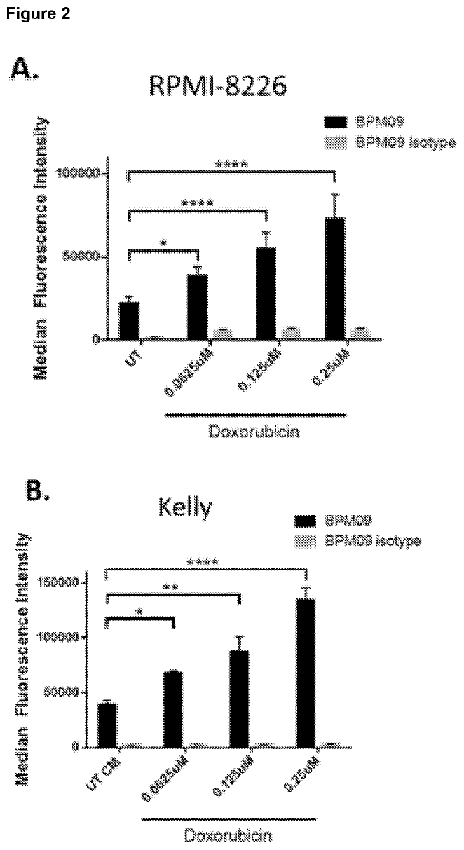

FIG. 1A shows that the myeloma cell line, RPMI-8226, is able to open the P2X7 pore in response to 0.5 mM BzATP whilst the neuroblastoma cell line Kelly is not. RPMI-8226 and Kelly cell lines were selected as representative models of blood born cancer (myeloma) and solid tumour (Neuroblastoma). FIGS. 1B, C and D shows the effect of increasing dose of chemotherapy (doxorubicin and 5 Fu) on RPMI-8226 and Kelly cells as measured using CellTitre-Blue (CTB) assay. These data were used to select the concentration of chemotherapy used in FIG. 2.

Doxorubicin also known as Adriamycin belongs to the family of anthracycline. It's mechanism of action is mediated by the blocking of topo isomerase 2 which results in inhibition of DNA replication, cell proliferation and ultimately cell death as observed in FIGS. 1B and C. 5 Fu also known as Fluorouracil is a chemotherapy which mechanism of action is mainly mediated by the inhibition of thymidylate synthase that in turn blocks synthesis of the pyrimidine thymidine, a nucleoside necessary for DNA replication. This result in the inhibition of cell proliferation and ultimately cell death as observed in FIG. 1D. Doxorubicin and 5 Fu chemotherapy were selected for their distinct mode of action.

### Example 2

Chemotherapy Treatment in Functional Myeloma RPMI-8226 and Non-Functional Neuroblastoma Kelly Cell Lines and Induction of nfP2X7 as Detected by BPM09.

Materials and Methods

500,000 cells were seeded in a 6 well plate and allowed to adhere over-night. Cells were then treated with increasing amount of chemotherapy or vehicle control for 72 hours to induce varying amount of cell killing. The remaining live cells were dissociated in PBS based enzyme-free dissociation buffer, washed and re-suspended in staining buffer (PBS, 2% FCS). Cells were then stained for 1 h with primary antibody raised against non-functional P2X7 (here 2-2-1hFc), washed 3 times in staining buffer prior to a 1 h incubation with fluorescently coupled secondary antibody and 7AAD. Fluorescence staining on live cells was acquired using a BD Accuri flow cytometer and analysed with FlowJo-flow cytometry analysis software. Median fluorescence intensity of the live cell population was analysed using 7AAD live dead staining.

Results

FIG. 2A to C shows that increasing amount of chemotherapy (doxorubicin and 5 Fu) on RPMI-8226 and Kelly cells leads to increased nfPX7 antibody binding (here 2-2-1hFc) in a dose dependent manner. This demonstrate that cancer cell lines with either functional P2X7 (RPMI-8226) or non-functional P2X7 (Kelly) show increased nfP2X7 antibody binding in response to various chemotherapy treatment (doxorubicin, 5 Fu, cisplatin, etc). These data support the rationale for combination therapy with chemotherapy and nfP2X7 targeted therapies.

### Example 3

Cell Lines with Acquired Resistance to Chemotherapy have Increased Expression of nfP2X7

Materials and Methods

A2780 parental cells, A2780 cells with acquired resistance to doxorubicin and A2780 cells with acquired resistance to cisplatin were cultured were obtained from ECACC repository (References: Proc Amer Assoc Cancer Res 1984; 25:336; Semin Oncol 1984; 11:285; Cancer Res 1987; 47:414; Cancer Res 1988; 48:5713).

500,000 A2780, A2780 with doxorubicin resistance and A2780 with cisplatin resistance cells were seeded in a 6 well plate and allowed to adhere over-night. Cells were dissociated in PBS based enzyme-free dissociation buffer, washed and re-suspended in staining buffer (PBS, 2% FCS). Cells were then stained for 1 h with primary antibody raised against non-functional P2X7 (here BPM09), washed 3 times in staining buffer prior to a 1 h incubation with fluorescently coupled secondary antibody and 7AAD. Fluorescence staining on live cells was acquired using a BD Accuri flow cytometer and analysed with FlowJo-flow cytometry analysis software. Median fluorescence intensity of the live cell population was analysed using 7AAD live dead staining.

Results

Experiments were conducted to determine the extent of binding of an antibody that specifically binds to nfP2X7 on live ovarian A2780 parental cells and A2780 cells which have acquired resistance to doxorubicin or cisplatin.

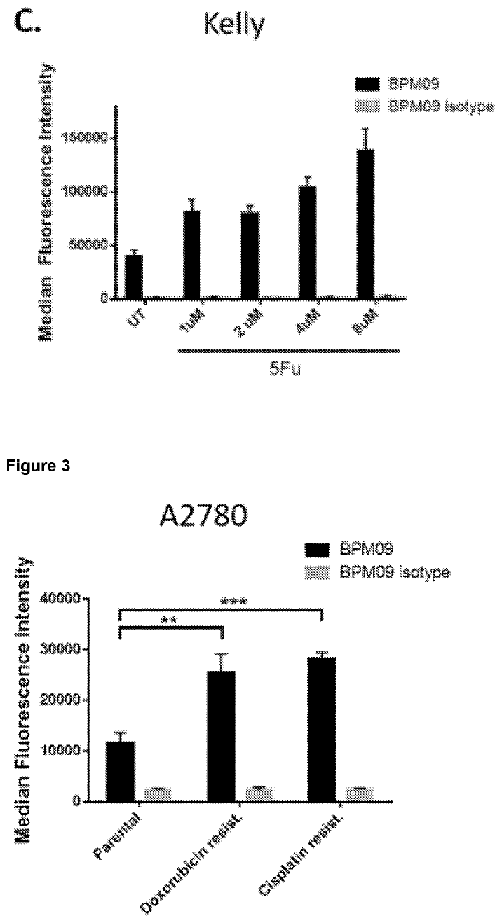

FIG. 3 shows a change in nfP2X7 antibody (here BPM09) binding to live ovarian A2780 parental cells and A2780 cells which have acquired resistance to Doxorubicin and Cisplatin. Data show that ovarian cancer cells that have acquired chemoresistance to doxorubicin and cisplatin have increased nfP2X7 antibody binding compared to parental A2780 cells.

These data demonstrate that the chemotherapy induced nfP2X7 increase is durable. Data also show that cells that have acquired chemoresistance have increased nfP2X7 exposure, which can be targeted by nfP2X7 directed therapeutics.

### Example 4

Patient Derived Xenografts Previously Treated with Chemotherapy and/or Radiotherapy have Increased Expression of nfP2X7

Materials and Methods

Immunohistochemistry was performed as previously described by Gilbert et al. Br J Dermatol. 2017. Five-micrometer thick sections were cut from formalin fixed paraffin embedded tissue from a TMA slides containing core biopsies of patient derived xenograft (PDX) models in duplicate. Heat-induced epitope retrieval was carried out prior to staining with primary mouse monoclonal anti-E200 antibody (BPM09) for 60 minutes at a final concentration of 1 μg/ml to 25 μg/ml and followed by Mach 4 mouse probe (Biocare, USA) for 15 minutes and Mach 4 universal polymer HRP for 25 minutes. Each step was separated by tissue rinsing for 5 minutes in Tris buffered saline. Dako liquid DAB was used as chromagen (5 minutes) and Haematoxylin (5 seconds) as counterstain. Slides were examined using a 20× objective. Intensity of the membranous staining has been scored by a pathologist.

Results

BPM09 membrane staining was scored after immunohistochemistry in a set of patient derived xenograft (PDX) models which were previously treated or not with chemotherapy and/or radiotherapy. The PDX model treated included in the study were colorectal cancer (16 samples), Testicular cancer (2 samples), Sarcoma (4 samples), Melanoma cancer (3 samples), Bladder cancer (1 sample), pancreatic cancer (2 samples), small cell lung cancer (1 sample), non-small cell lung cancer (2 samples), Ovarian cancer (1 sample), Uterine Cervix cancer (2 samples) and Breast cancer (6 samples). Various chemotherapy regimen were used including the following chemotherapy as single therapy or in combination: 5 FU, Folinic acid, Bleomycin, Etoposide, Cisplatin, Capecitabin, Oxaliplatin, Dacarbazine, Cyclophosphamide, Vincristine, Doxorubicin, Irinotecan, Gemcitabine, Mitomycin-C, Gemcitabine, Carboplatin, Paclitaxel, pemetrexed, hydroxyethyl-chloroethyl nitrosourea (HeCNU), Tamoxifen, Methotrexat, Epirubicin, Vindesine, Erlotinib, Bevacizumab, Cetuximab and Radiotherapy.

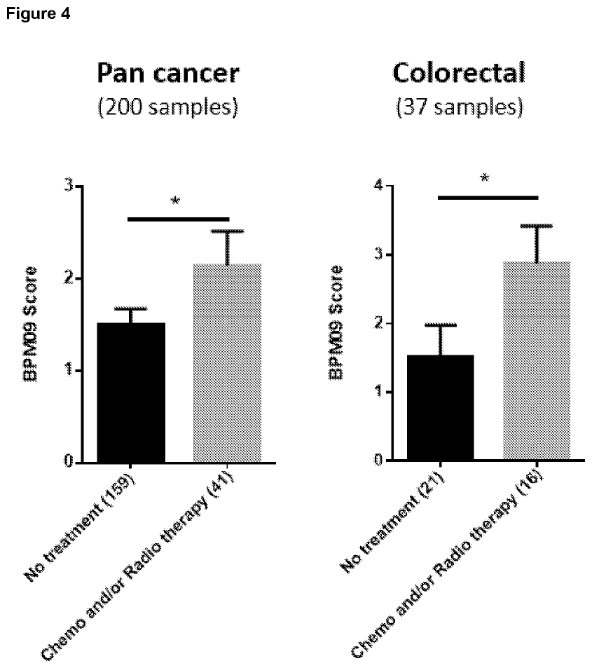

FIG. 4 shows that patient derived xenograft previously treated with chemotherapy and/or radiotherapy (multiple chemotherapy regiment and other treatments such as radiotherapy were pooled in this analysis) have increased nfP2X7 antibody binding (here BPM09). These data demonstrate that multiple non-targeted therapies regimen including chemotherapy and radiotherapy lead to increased nfP2X7 exposure at the surface of tumour cells and support the use of nfP2X7 targeted therapies in patient that have previously been treated with such non-targeted treatment.

### Example 5

Cancer Cells Pre-Treated with Chemotherapy are Sensitive to Targeting of nfP2X7

Materials and Methods

Kelly cells, untreated or treated for 72 hours with 4 uM 5-FU were seeded at 50,000 cells per well in black clear bottom 96 well plate and allowed to attach overnight. Cells were then loaded with 6.6 ug/ml Calcein in pre-warmed media for 30 minutes. After loading, cells were washed 3 times with serum free RPMI 1640 and then 5 ul mouse serum from mice immunised with PBS or BIL06v was added in 50 ul serum free RPMI 1640 per well (sera from 4 PBS immunised mice and 16 mice BIL06v immunised mice in individual wells). Cells were incubated on ice for 30 minutes. 50 ul of 80% baby rabbit complement (Cedar Lane—cl3441-s50) in pre-warmed serum free RPMI1640 was added to each well. Plate was incubated at 37° C. for 2 hours. After 2 hours media was transferred to V bottom plate and centrifugated at 2000 g for 5 minutes to pellet any cells. 80 ul of supernatant per well transferred to clear bottom black 96 well plate. Calcein fluorescence was measured at 495 nm/515 nm. Data shown is Calcein fluorescence from wells containing sera from BIL06v immunised mice on untreated or 5-FU pre-treated cells, normalised to average Calcein fluorescence from wells containing sera from PBS immunised mice on cells with same pre-treatment

Results

Effect of 5 Fu on nfP2X7 antibodies (here polyclonal mouse antibody) mediated complement dependent cytotoxicity on Kelly cells.

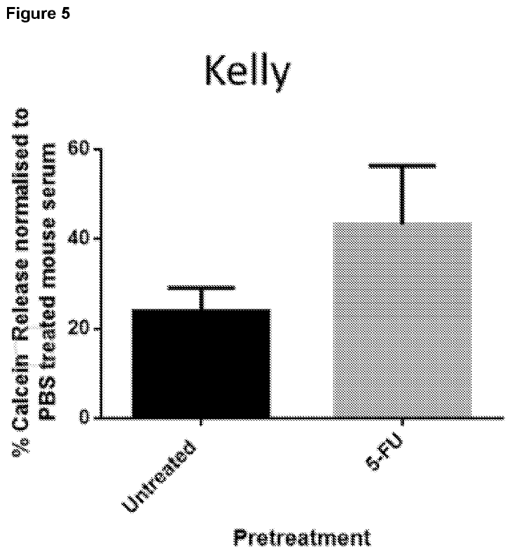

Data demonstrate that combination therapy between chemotherapy and nfP2X7 targeted therapies could be undertaken to promote anti-tumour effects as demonstrated by the increased complement dependent cytotoxicity nfP2X7 targeted antibodies against Kelly cells treated with chemotherapy (FIG. 5). Overall, the data show across various chemotherapy treatments (doxorubicin, 5 Fu, cisplatin, etc) and cancer model (myeloma, neuroblastoma, ovarian, colorectal) that chemotherapy treatment lead to an increased levels of nfP2X7 at the surface of cancer cells which can be used as a rationale for combination therapy with nfP2X7 targeted antibodies.

### Example 6

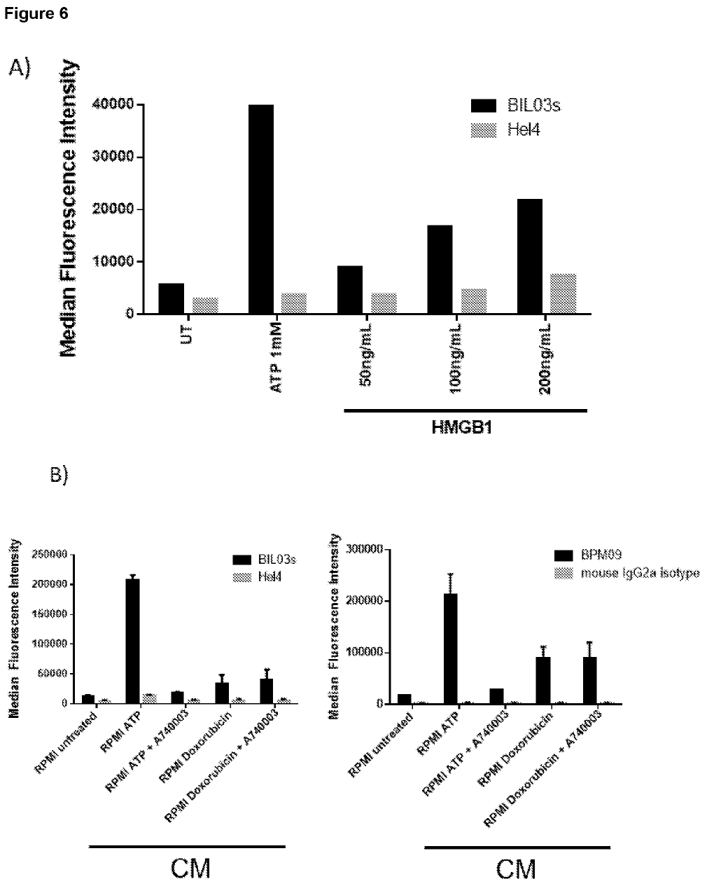

Data shown in FIG. 6a indicates that DAMPs such as HMGB1 are likely to mediate the effect. Both chemo- and radio-therapy drive the release of DAMPs such as HMGB1 which in itself drives the increase in nfP2X7.

FIG. 6b describes data showing that P2X7 inhibitor (A 740003-N-[1-[[(Cyanoamino)(5-quinolinylamino)methylene]amino]-2,2-dimethylpropyl]-3,4-dimethoxybenzeneacetamide, Cat. No. 3701, Tocris) does not block the conditioned media induced nfP2X7 induction. This shows that, surprisingly, ATP mediated activation of P2X7 receptor is not mediating the chemotherapy effect.

1. A method of treating cancer in an individual who has not responded, or no longer responds, to chemotherapy and/or radiotherapy, the method comprising
providing an individual who has not responded, or no longer responds, to a chemotherapeutic agent and/or radiotherapy;
administering a P2X7 receptor targeted therapy to the individual;
wherein the P2X7 receptor has an impaired response to ATP such that it is unable to form an apoptotic pore under normal physiological conditions,
thereby treating cancer in the individual.

2. A method according to claim 1, wherein the P2X7 receptor targeted therapy is a molecule that binds to a P2X7 receptor has an impaired response to ATP such that it is unable to form an apoptotic pore under normal physiological conditions.

3. A method according to claim 1, wherein the P2X7 receptor targeted therapy is a molecule that induces an immune response to a P2X7 receptor has an impaired response to ATP such that it is unable to form an apoptotic pore under normal physiological conditions.

4. A method according to claim 1, wherein the P2X7 receptor targeted therapy is a molecule that reduces the level of a P2X7 receptor has an impaired response to ATP such that it is unable to form an apoptotic pore under normal physiological conditions.

5. A method according to claim 2, wherein the molecule that binds to a P2X7 receptor is an antibody or cell-based therapy.

6. A method according to claim 3, wherein the molecule that induces an immune response to P2X7 receptor is an immunogen in the individual in the form of a P2X7 receptor, or a fragment of a P2X7 receptor that is capable of inducing an immune response to a P2X7 receptor in the individual, wherein the P2X7 receptor has an impaired response to ATP such that it is unable to form an apoptotic pore under normal physiological conditions.

7. A method according to claim 4, wherein the molecule that reduces the level of P2X7 receptor is an interfering RNA.

8. A method of treating cancer in an individual who has not responded, or no longer responds, to chemotherapy and/or radiotherapy, the method comprising
providing an individual who has not responded, or no longer responds, to a chemotherapeutic agent;
providing in the individual a whole antibody or a fragment thereof including a variable domain for binding to a P2X7 receptor that is expressed by the individual;
wherein the P2X7 receptor has an impaired response to ATP such that it is unable to form an apoptotic pore under normal physiological conditions,
thereby treating cancer in the individual.

9. The method of any one of claims 1 to 8, wherein the antibody fragment is selected from the group consisting of a dAb, Fab, Fd, Fv, F(ab′)2 and scFv.

10. The method of any one of claims 1 to 9, wherein the antibody or fragment thereof does not bind to functional P2X7 receptors.

11. The method of any one of claims 1 to 10, wherein antibody or fragment thereof binds to the amino acid sequence as set forth in any one of SEQ ID NOs: 1 to 11.

12. The method according to claim 11, wherein antibody or fragment thereof binds to the amino acid sequence as set forth in any one of SEQ ID NO: 2 to 5.

13. A method of treating cancer in an individual who has not responded, or no longer responds, to chemotherapy and/or radiotherapy, the method comprising
providing an individual who has not responded, or no longer responds, to a chemotherapeutic agent;
providing in the individual a cell-based therapy that targets P2X7 receptor expressing cancer cells;
wherein the P2X7 receptor has an impaired response to ATP such that it is unable to form an apoptotic pore under normal physiological conditions,
thereby treating cancer in the individual.

14. A method according to claim 13, wherein the cell-based therapy that targets P2X7 receptor expressing cancer cells is a cytotoxic cell that has the capacity to bind to a P2X7 receptor expressing cancer cell.

15. A method according to claim 14, wherein the cytotoxic cell that has the capacity to bind to a P2X7 receptor expressing cancer cell is a CAR-T cell.

16. A method according to any one of claims 13 to 15, wherein the cytotoxic cell, preferably a CAR-T cell, expresses a chimeric antigen receptor including an antigen-recognition domain and a signalling domain, wherein the antigen-recognition domain recognises a dysfunctional or non-functional P2X7 receptor (i.e. a P2X7 receptor has an impaired response to ATP such that it is unable to form an apoptotic pore under normal physiological conditions).

17. A method according to claim 16, wherein the dysfunctional or non-functional P2X7 receptor has a reduced capacity to bind ATP compared to an ATP-binding capacity of a wild-type (functional) P2X7 receptor.

18. A method according to claim 16 or 17, wherein the dysfunctional or non-functional P2X7 receptor has a conformational change that renders the receptor dysfunctional or non-functional.

19. A method according to claim 18, wherein the antigen-recognition domain recognises an epitope that includes one or more amino acid residues spanning from glycine at amino acid position 200 to cysteine at amino acid position 216 of the dysfunctional P2X7 receptor.

20. A method according to any one of claims 16 to 19, wherein the antigen-recognition domain comprises amino acid sequence homology to the amino acid sequence of an antibody, or a fragment thereof, that binds to the dysfunctional or non-functional P2X7 receptor, including any antibody, or fragment thereof, as described herein.

21. A method according to any one of claims 16 to 20, wherein the antigen-recognition domain comprises amino acid sequence homology to the amino acid sequence of a fragment-antigen binding (Fab) portion, a single-chain variable fragment (scFv), or a single-antibody domain (dAb) of an antibody that binds to a dysfunctional or non-functional P2X7 receptor.

22. A method according to any one of claims 16 to 21, wherein the antigen-recognition domain comprises amino acid sequence homology to the amino acid sequence of a multivalent single-chain variable fragment (scFv) that binds to a dysfunctional P2X7 receptor.

23. A method according to claim 22, wherein the multivalent single-chain variable fragment (scFv) may be di-valent or tri-valent scFv.

24. A method according to any one of claims 16 to 23, wherein the signalling domain may include a portion derived from an activation receptor.

25. A method according to claim 24, wherein the activation receptor is a member of the CD3 co-receptor complex.

26. A method according to claim 25, wherein the portion derived from the CD3 co-receptor complex is CD3-zeta.

27. A method according to claim 25, wherein the activation receptor is an Fc receptor, preferably the portion derived from the Fc receptor is Fc epsilon RI or Fc gamma RI.

28. A method according to any one of claims 16 to 27, wherein the signalling domain comprises a portion derived from a co-stimulatory receptor.

29. A method according to claim 28, wherein the signalling domain comprises a portion derived from an activation receptor and a portion derived from a co-stimulatory receptor.

30. A method according to claim 28 or 29, wherein the co-stimulatory receptor is selected from the group consisting of CD27, CD28, CD30, CD40, DAP10, OX40, 4-1 BB (CD137) and ICOS.

31. A method according to any one of claims 13 to 30, wherein the cytotoxic cell is any one of:
a leukocyte,
a Peripheral Blood Mononuclear Cell (PBMC),
a lymphocyte,
a T cell,
a CD4+ T cell,
a CD8+ T cell,
a natural killer cell, or
a natural killer T cell.

32. A method of treating cancer in an individual who has not responded, or no longer responds, to chemotherapy and/or radiotherapy, the method comprising
providing an individual who has not responded, or no longer responds, to chemotherapy;
forming an immune response in the individual to a P2X7 receptor that is expressed by the individual;
wherein the P2X7 receptor has an impaired response to ATP such that it is unable to form an apoptotic pore under normal physiological conditions,
thereby treating cancer in the individual.

33. A method of claim 32, wherein the immune response is formed by providing an immunogen in the individual in the form of a P2X7 receptor, or a fragment of a P2X7 receptor that is capable of inducing an immune response to a P2X7 receptor in the individual, wherein the P2X7 receptor has an impaired response to ATP such that it is unable to form an apoptotic pore under normal physiological conditions.

34. A method of claim 32 or 33, wherein the fragment of a P2X7 receptor has an amino acid sequence as set forth in any one of SEQ ID NOs: 1 to 10.

35. A method of any one of claims 32 to 34, wherein the immunogen is provided in an initial administration to the individual, thereby forming a response that includes IgM production in the individual.

36. A method of any one of claims 32 to 35, wherein the immunogen is provided in an initial administration to the individual, thereby forming a response that includes IgM production, and at a later time, in a further administration to the initial administration, thereby forming a response that includes IgG production.

37. A method of any one of claims 1 to 36, wherein the chemotherapy that the individual has not responded, or no longer responds, to is one or more chemotherapeutic agents selected from the group consisting of oxazaphosphorines, topoisomerase I inhibitors, topoisomerase II inhibitors, proteasome inhibitors, antifolates, nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, purine antagonists, antimetabolites, antibiotics, epipodophyllotoxins, platinum based agents, ribonucleotide reductase inhibitors, vinca alkaloids, substituted ureas, hydrazine derivatives, adrenocortical suppressants, endostatin, camptothecins, oxaliplatin, doxorubicin and doxorubicin analogs, antibiotics, L-asparaginase, tyrosine kinase inhibitors, and derivatives or variants thereof.

38. A method of any one of claims 1 to 36, wherein the chemotherapy that the individual has not responded, or no longer responds, to is one or more chemotherapeutic agents selected from the group consisting of doxorubicin, cisplatin, vincristine, dacarbazine (DTIC), cyclophosphamide, CPT-11, oxaliplatin, gemcitabine and 5-fluorouracil/leucovorin.

39. A method of any one of claims 1 to 36, wherein the chemotherapy that the individual has not responded, or no longer responds, to is one or more chemotherapeutic agents selected from the group consisting of 5 FU, Folinic acid, Bleomycin, Etoposide, Cisplatin, Capecitabin, Oxaliplatin, Dacarbazine, Cyclophosphamide, Vincristine, Doxorubicin, Irinotecan, Gemcitabine, Mitomycin-C, Gemcitabine, Carboplatin, Paclitaxel, pemetrexed, hydroxyethyl-chloroethyl nitrosourea (HeCNU), Tamoxifen, Methotrexat, Epirubicin, Vindesine, Erlotinib, Bevacizumab, and Cetuximab.

40. A method of treating cancer in an individual, the method comprising
administering a chemotherapeutic agent and/or radiotherapy to the individual in whom the cancer is to be treated; and
administering to the individual a whole antibody or a fragment thereof including a variable domain for binding to a P2X7 receptor that is expressed by the individual;
wherein the P2X7 receptor has an impaired response to ATP such that it is unable to form an apoptotic pore under normal physiological conditions,
thereby treating cancer in the individual.

41. A method of claim 40, wherein the chemotherapeutic agent or radiotherapy, and antibody or fragment thereof are administered simultaneously.

42. A method of claim 41, wherein the chemotherapeutic agent or radiotherapy, and antibody or fragment thereof are administered sequentially.

43. A method of claim 42, wherein the chemotherapeutic agent or radiotherapy, is administered prior to the antibody or fragment thereof.

44. A method of treating cancer in an individual, the method comprising
administering a chemotherapeutic agent and/or radiotherapy to the individual in whom the cancer is to be treated; and
forming an immune response in the individual to a P2X7 receptor that is expressed by the individual;
wherein the P2X7 receptor has an impaired response to ATP such that it is unable to form an apoptotic pore under normal physiological conditions,
thereby treating cancer in the individual.

45. A method of claim 44, wherein the immune response is formed by providing an immunogen in the individual in the form of a P2X7 receptor, or a fragment of a P2X7 receptor that is capable of inducing an immune response to a P2X7 receptor in the individual, wherein the P2X7 receptor has an impaired response to ATP such that it is unable to form an apoptotic pore under normal physiological conditions.

46. A method of claim 44 or 45, wherein the fragment of a P2X7 receptor has an amino acid sequence as set forth in any one of SEQ ID NOs: 1 to 10.

47. A method of any one of claims 44 to 46, wherein the immunogen is provided in an initial administration to the individual, thereby forming a response that includes IgM production in the individual.

48. A method of any one of claims 44 to 47, wherein the immunogen is provided in an initial administration to the individual, thereby forming a response that includes IgM production, and at a later time, in a further administration to the initial administration, thereby forming a response that includes IgG production.

49. A method of any one of claims 1 to 48, wherein the cancer is selected from brain cancer, oesophageal cancer, mouth cancer, tongue cancer, thyroid cancer, lung cancer, stomach cancer, pancreatic cancer, kidney cancer, colorectal cancer, rectal cancer, prostate cancer, bladder cancer, cervical cancer, epithelial cell cancers, skin cancer, leukaemia, lymphoma, myeloma, breast cancer, ovarian cancer, endometrial cancer and testicular cancer.

50. The method according to any one of claims 1 to 49, wherein the cancer is selected from lung cancer, oesophageal cancer, ovarian cancer, stomach cancer, colorectal cancer, prostate cancer, bladder cancer, cervical cancer, vaginal cancers, epithelial cell cancers, skin cancer, blood-related cancers, breast cancer, endometrial cancer, uterine cancer and testicular cancer.

51. The method of any one of claims 1 to 50, wherein the individual has neuroblastoma.

52. The method of any one of claims 1 to 50, wherein the individual has ovarian cancer.

53. The method of any one of claims 1 to 50, wherein the individual has colorectal cancer.


SEQUENCE LISTING (13 sequences)

SEQ ID NO: 1 - protein, 595 aa, Homo sapiens [sequence omitted]

SEQ ID NO: 2 - protein, 9 aa, Homo sapiens
HNYTTRNIL

SEQ ID NO: 3 - protein, 10 aa, Homo sapiens
GHNYTTRNIL

SEQ ID NO: 4 - protein, 13 aa, Homo sapiens
DFPGHNYTTRNIL

SEQ ID NO: 5 - protein, 17 aa, Homo sapiens
GHNYTTRNILPGLNITC

SEQ ID NO: 6 - protein, 209 aa, Homo sapiens
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVHEGNQKTCEVSAWCPIEAVEEAPRPALLNSAENFTVLIKNNIDFPGHNYTTRNIL

SEQ ID NO: 7 - protein, 49 aa, Homo sapiens
KTTNVSLYPGYNFRYAKYYKENNVEKRTLIKVFGIRFDILVFGTGGKFD

SEQ ID NO: 8 - protein, 17 aa, Homo sapiens
KYYKENNVEKRTLIKVF

SEQ ID NO: 9 - protein, 11 aa, Homo sapiens
GHNYTTRNILP

SEQ ID NO: 10 - protein, 11 aa, Homo sapiens
AKYYKENNVEK

SEQ ID NO: 11 - protein, 25 aa, Homo sapiens
GHNYTTRNILPGAGAKYYKENNVEK

SEQ ID NO: 12 - DNA, 25 bp, Homo sapiens
gagatattgtgaggacaaattgaga

SEQ ID NO: 13 - DNA, 25 bp, Homo sapiens
cccgcagagcaaaggaattcagacc